SEQUENCE LISTING (18 sequences)

SEQ ID NO: 1 - DNA, 2077 bp, Homo sapiens
  ref <301>: AUTHORS: Rezaie, T., Child, A., Hitchings, R., Brice, G., Miller, L., Coca-Prodos, M., Heon, E., Krupin, T., Ritch, R., Kreutzer, D., Crick, R.P., and Sarfarazi, M.
  ref <302>: TITLE: Adult-Onset Primary Open-Angle Glaucoma Caused by Mutations in Optineurin
  ref <303>: JOURNAL: Science
  ref <304>: VOLUME: 295
  ref <305>: ISSUE: 5557
  ref <306>: PAGES: 1077-1079
  ref <307>: DATE: 2002
  ref <308>: DATABASE ACCESSION NUMBER: AF420371
  ref <309>: DATABASE ENTRY DATE: 2002-02-11
  ref <313>: RELEVANT RESIDUES: (1)..(2077)
atcccggtcgggagttctctccaggcggcacgatgccgaggaaacagtgaccctgagcgaagccaagccgggcggcaggtgtggctttgatagctggtggtgccacttcctggccttggatgagccgtacgcctctgtaaacccaacttcctcacctttgaaacagctgcctggttcagcattaatgaagattagtcagtgacaggcctggtgtgctgagtccgcacatagaagaatcaaaaatgtccaaaatgtaactggagagaaagtgggcaacttttggagtgacttttccacaggaacttctgcaatgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgaaggagaagcagaagggtcagtaaaagaaatcaagcatagtcctgggcccacgagaacagtctccactggcacggcattgtctaaatataggagcagatctgcagatggggccaagaattacttcgaacatgaggagttaactgtgagccagctcctgctgtgcctaagggaagggaatcagaaggtggagagacttgaagttgcactcaaggaggccaaagaaagagtttcagattttgaaaagaaaacaagtaatcgttctgagattgaaacccagacagaggggagcacagagaaagagaatgatgaagagaaaggcccggagactgttggaagcgaagtggaagcactgaacctccaggtgacatctctgtttaaggagcttcaagaggctcatacaaaactcagcgaagctgagctaatgaagaagagacttcaagaaaagtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtccaaattaactgtgctacagatgacacacaacaagcttcttcaagaacataataatgcattgaaaacaattgaggaactaacaagaaaagagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcagatggaagtttactgttctgattttcatgctgaaagagcagcgagagagaaaattcatgaggaaaaggagcaactggcattgcagctggcagttctgctgaaagagaatgatgctttcgaagacggaggcaggcagtccttgatggagatgcagagtcgtcatggggcgagaacaagtgactctgaccagcaggcttaccttgttcaaagaggagctgaggacagggactggcggcaacagcggaatattccgattcattcctgccccaagtgtggagaggttctgcctgacatagacacgttacagattcacgtgatggattgcatcatttaagtgttgatgtatcacctccccaaaactgttggt

SEQ ID NO: 2 - protein, 577 aa, Homo sapiens
  ref <301>: AUTHORS: Rezaie et al.
  ref <302>: TITLE: Adult-Onset Primary Open-Angle Glaucoma Caused by Mutations in Optineurin
  ref <303>: JOURNAL: Science
  ref <304>: VOLUME: 295
  ref <305>: ISSUE: 5557
  ref <306>: PAGES: 1077-1079
  ref <307>: DATE: 2002
  ref <308>: DATABASE ACCESSION NUMBER: AF420371
  ref <309>: DATABASE ENTRY DATE: 2002-02-11
  ref <313>: RELEVANT RESIDUES: (1)..(577)
MSHQPLSCLTEKEDSPSESTGNGPPHLAHPNLDTFTPEELLQQMKELLTENHQLKEAMKLNNQAMKGRFEELSAWTEKQKEERQFFEIQSKEAKERLMALSHENEKLKEELGKLKGKSERSSEDPTDDSRLPRAEAEQEKDQLRTQVVRLQAEKADLLGIVSELQLKLNSSGSSEDSFVEIRMAEGEAEGSVKEIKHSPGPTRTVSTGTALSKYRSRSADGAKNYFEHEELTVSQLLLCLREGNQKVERLEVALKEAKERVSDFEKKTSNRSEIETQTEGSTEKENDEEKGPETVGSEVEALNLQVTSLFKELQEAHTKLSEAELMKKRLQEKCQALERKNSAIPSELNEKQELVYTNKKLELQVESMLSEIKMEQAKTEDEKSKLTVLQMTHNKLLQEHNNALKTIEELTRKESEKVDRAVLKELSEKLELAEKALASKQLQMDEMKQTIAKQEEDLETMTILRAQMEVYCSDFHAERAAREKIHEEKEQLALQLAVLLKENDAFEDGGRQSLMEMQSRHGARTSDSDQQAYLVQRGAEDRDWRQQRNIPIHSCPKCGEVLPDIDTLQIHVMDCII

SEQ ID NO: 3 - DNA, 1856 bp, Homo sapiens
  ref <301>: AUTHORS: Rezaie et al.
  ref <302>: TITLE: Adult-Onset Primary Open-Angle Glaucoma Caused by Mutations in Optineurin
  ref <303>: JOURNAL: Science
  ref <304>: VOLUME: 295
  ref <305>: ISSUE: 5557
  ref <306>: PAGES: 1077-1079
  ref <307>: DATE: 2002
  ref <308>: DATABASE ACCESSION NUMBER: AF420372
  ref <309>: DATABASE ENTRY DATE: 2002-02-11
  ref <313>: RELEVANT RESIDUES: (1)..(1856)
atcccggtcgggagttctctccaggcggcacgatgccgaggaaacagtgaccctgagcgaagccaagccgggcggcaggaacttctgcaatgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgaaggagaagcagaagggtcagtaaaagaaatcaagcatagtcctgggcccacgagaacagtctccactggcacggcattgtctaaatataggagcagatctgcagatggggccaagaattacttcgaacatgaggagttaactgtgagccagctcctgctgtgcctaagggaagggaatcagaaggtggagagacttgaagttgcactcaaggaggccaaagaaagagtttcagattttgaaaagaaaacaagtaatcgttctgagattgaaacccagacagaggggagcacagagaaagagaatgatgaagagaaaggcccggagactgttggaagcgaagtggaagcactgaacctccaggtgacatctctgtttaaggagcttcaagaggctcatacaaaactcagcgaagctgagctaatgaagaagagacttcaagaaaagtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtccaaattaactgtgctacagatgacacacaacaagcttcttcaagaacataataatgcattgaaaacaattgaggaactaacaagaaaagagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcagatggaagtttactgttctgattttcatgctgaaagagcagcgagagagaaaattcatgaggaaaaggagcaactggcattgcagctggcagttctgctgaaagagaatgatgctttcgaagacggaggcaggcagtccttgatggagatgcagagtcgtcatggggcgagaacaagtgactctgaccagcaggcttaccttgttcaaagaggagctgaggacagggactggcggcaacagcggaatattccgattcattcctgccccaagtgtggagaggttctgcctgacatagacacgttacagattcacgtgatggattgcatcatttaagtgttgatgtatcacctccccaaaactgttggt

SEQ ID NO: 4 - protein, 577 aa, Homo sapiens; identical to SEQ ID NO: 2
  ref <301>: AUTHORS: Rezaie et al.
  ref <302>: TITLE: Adult-Onset Primary Open-Angle Glaucoma Caused by Mutations in Optineurin
  ref <303>: JOURNAL: Science
  ref <304>: VOLUME: 295
  ref <305>: ISSUE: 5557
  ref <306>: PAGES: 1077-1079
  ref <307>: DATE: 2002
  ref <308>: DATABASE ACCESSION NUMBER: AF420372
  ref <309>: DATABASE ENTRY DATE: 2002-02-11
  ref <313>: RELEVANT RESIDUES: (1)..(577)

SEQ ID NO: 5 - DNA, 2008 bp, Homo sapiens
  ref <301>: AUTHORS: Rezaie et al.
  ref <302>: TITLE: Adult-Onset Primary Open-Angle Glauconma Caused by Mutations in Optineurin
  ref <303>: JOURNAL: Science
  ref <304>: VOLUME: 295
  ref <305>: ISSUE: 5557
  ref <306>: PAGES: 1077-1079
  ref <307>: DATE: 2002
  ref <308>: DATABASE ACCESSION NUMBER: AF420373
  ref <309>: DATABASE ENTRY DATE: 2002-02-11
  ref <313>: RELEVANT RESIDUES: (1)..(2008)
atcccggtcgggagttctctccaggcggcacgatgccgaggaaacagtgaccctgagcgaagccaagccgggcggcaggtgtggctttgatagctggtggtgccacttcctggccttggatgagccgtacgcctctgtaaacccaacttcctcacctttgaaacagctgcctggttcagcattaatgaagattagtcagtgacaggcctggtgtgctgagtccgcacatagaacttctgcaatgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgaaggagaagcagaagggtcagtaaaagaaatcaagcatagtcctgggcccacgagaacagtctccactggcacggcattgtctaaatataggagcagatctgcagatggggccaagaattacttcgaacatgaggagttaactgtgagccagctcctgctgtgcctaagggaagggaatcagaaggtggagagacttgaagttgcactcaaggaggccaaagaaagagtttcagattttgaaaagaaaacaagtaatcgttctgagattgaaacccagacagaggggagcacagagaaagagaatgatgaagagaaaggcccggagactgttggaagcgaagtggaagcactgaacctccaggtgacatctctgtttaaggagcttcaagaggctcatacaaaactcagcgaagctgagctaatgaagaagagacttcaagaaaagtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtccaaattaactgtgctacagatgacacacaacaagcttcttcaagaacataataatgcattgaaaacaattgaggaactaacaagaaaagagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcagatggaagtttactgttctgattttcatgctgaaagagcagcgagagagaaaattcatgaggaaaaggagcaactggcattgcagctggcagttctgctgaaagagaatgatgctttcgaagacggaggcaggcagtccttgatggagatgcagagtcgtcatggggcgagaacaagtgactctgaccagcaggcttaccttgttcaaagaggagctgaggacagggactggcggcaacagcggaatattccgattcattcctgccccaagtgtggagaggttctgcctgacatagacacgttacagattcacgtgatggattgcatcatttaagtgttgatgtatcacctccccaaaactgttggt

SEQ ID NO: 6 - protein, 577 aa, Homo sapiens; identical to SEQ ID NO: 2
  ref <301>: AUTHORS: Rezaie et al.
  ref <302>: TITLE: Adult Onset Primary Open-Angle Glaucoma Caused by Mutations in Optineurin
  ref <303>: JOURNAL: Science
  ref <304>: VOLUME: 295
  ref <305>: ISSUE: 5557
  ref <306>: PAGES: 1077-1079
  ref <307>: DATE: 2002
  ref <308>: DATABASE ACCESSION NUMBER: AF420272
  ref <309>: DATABASE ENTRY DATE: 2002-02-11
  ref <313>: RELEVANT RESIDUES: (1)..(577)

SEQ ID NO: 7 - protein, 18 aa, Homo sapiens
MSHQPLSCLTEKEDSPSE

SEQ ID NO: 8 - protein, 18 aa, Homo sapiens
EVLPDIDTLQIHVMDCII

SEQ ID NO: 9 - DNA, 2023 bp, Mus musculus
  ref <308>: DATABASE ACCESSION NUMBER: AY071834
  ref <309>: DATABASE ENTRY DATE: 2002-07-06
  ref <313>: RELEVANT RESIDUES: (1)..(2023)
cgggtcgcgaggccgcgcgtcaccgtccagctgcccggtcgccgccccccggctggtccctggcgggcgtcggcatccagagctttgggacaaaataccaacgaagcagtgacctaagaggagaggatccctgtggccggacctgttaccatgtcccatcaacctctgagctgcctgactgagaagggggacagcccttgtgagaccccaggaaatggaccctccaatatggttcaccccagcctggacacattcacccctgaggagctgctgcagcaaatgaaggaactcctggttgagaaccaccagctgaaagaagccatgaagctaaataatcaagctatgaaagggcgatttgaggagctgtccgcctggacagagaagcagaaggaagagcgcctgttgtttgagatgcaaagcaaagaggttaaggagcgccttaaggccctgactcatgaaaatgagaggctgaaggaagagcttggaaaattcaaagagaaatcagaaaagccattggaagacctcacaggtggctacaggtatcccagagccttggaggaggaagtggagaagctgaagacccaggtggagcaggaagtggagcatctgaagatccaggtgatgcgccttcgggctgaaaaggcagacctgctgggcatcgtctcagaactgcagctcaaactcaactccggcggctcctcggaagactccttcgttgagatcaggatgaccgaaggagagactgaaggggcaatgaaggagatgaagaactgccctacacccacaagaacagaccccatcagcttgagcaactgtacagaggatgccaggagttgtgcggagtttgaagaactgactgtgagccagcttctgctttgcctaagggaaggaaaccaaaaggtggagagacttgaagtcgccctcagagaagccaaagaaagaatttcagattttgaaaagaaagcaaatggccattcttctactgagaagcagacagcgaggagagcagacagagagaaggaggacaaaggccaagagagtgttggaagcgaagtggaaacactgagcattcaagtgacctctctgtttaaggagcttcaagaggcacacacaaaactcagtgaggctgagctgatgaagaagagacttcaagaaaagtgtcaggctctggagaggaagaactctgcaacaccatcagagctgaatgaaaagcaagagctcgtttacagtaacaagaagttagagctgcaggtggagagcatgcgctccgaaatcaagatggagcaggccaagacagaggaggagaagtccaggttagccactctgcaggcaactcacaacaagctccttcaagaacataataaggcactgaaaacaattgaagaactaaccaagcaacaggcagaaaaggtggacaagatgttgctgcaggagctcagcgagaagctggagctggcagagcaggctctggcatccaaacagctccagatggatgagatgaagcagacgctcgctaagcaggaggaagacctggagaccatggccgtcctcagggctcagatggaggtgtactgctcagattttcacgctgagagagcagcaagagagaagattcatgaagaaaaggagcagctggccttgcagctcgcgattttgctgaaagagaacaatgacattgaagagggaggcagtagacagtccctgatggaaatgcagtgccgacacggggcaagaaccagtgactctgaccagcagacttacctgtttcaaagaggagccgaggacaggagctggcagcacgggcagcagcctcgcagtattccgattcactcctgccccaagtgcggggaggtcctgccggacatcgacacgcttcagatccatgtgatggactgcatcatttgagtgttctctccagtccccaaagctcttggtaaatgccagatttctcctccaacacttgtacttctgtcttctttattgtcacatatatcacaaatatttttgacacactgtacccctc

SEQ ID NO: 10 - protein, 584 aa, Mus musculus
  ref <308>: DATABASE ACCESSION NUMBER: AY071834
  ref <309>: DATABASE ENTRY DATE: 2002-07-06
  ref <313>: RELEVANT RESIDUES: (1)..(584)
MSHQPLSCLTEKGDSPCETPGNGPSNMVHPSLDTFTPEELLQQMKELLVENHQLKEAMKLNNQAMKGRFEELSAWTEKQKEERLLFEMQSKEVKERLKALTHENERLKEELGKFKEKSEKPLEDLTGGYRYPRALEEEVEKLKTQVEQEVEHLKIQVMRLRAEKADLLGIVSELQLKLNSGGSSEDSFVEIRMTEGETEGAMKEMKNCPTPTRTDPISLSNCTEDARSCAEFEELTVSQLLLCLREGNQKVERLEVALREAKERISDFEKKANGHSSTEKQTARRADREKEDKGQESVGSEVETLSIQVTSLFKELQEAHTKLSEAELMKKRLQEKCQALERKNSATPSELNEKQELVYSNKKLELQVESMRSEIKMEQAKTEEEKSRLATLQATHNKLLQEHNKALKTIEELTKQQAEKVDKMLLQELSEKLELAEQALASKQLQMDEMKQTLAKQEEDLETMAVLRAQMEVYCSDFHAERAAREKIHEEKEQLALQLAILLKENNDIEEGGSRQSLMEMQCRHGARTSDSDQQTYLFQRGAEDRSWQHGQQPRSIPIHSCPKCGEVLPDIDTLQIHVMDCII

SEQ ID NO: 11 - DNA, 1925 bp, Macaca mulatta
  ref <308>: DATABASE ACCESSION NUMBER: AY228373
  ref <309>: DATABASE ENTRY DATE: 2002-03-30
  ref <313>: RELEVANT RESIDUES: (1)..(1925)
atcccggtcgggagttctctccaggcggcacgatgccaaggaagcagtgaccctgagcgaagccaagccgggcggcaggaacttctgcaatgtcccatcaacctctcagctgcctcactgaaaagggggacagccccagtgaaagcacaggaaatggacccccccatctggcccacccaaacctggacacattcaccccagaggagctgctgcagcagatgaaagagctcctgaccgagaaccaccagctgaaagaagccatgaagctaaataaccaagccatgaaagggcggtttgaggagctttcggcctggacagagaaacagaaggaagaacgccagttttttgagacacagagcaaagaagccaaagagcgtctcatggccttgagtcacgagaatgagaaattgaaggaagagcttggaaaactgaaagggaaatcagaaaggtcatctgaggaccccactgatgactccaggcttcccagggcagaagcggagcaggaaaaggaccagctcaggacccaggtgacgaggctacaagcagagaaggcagacctgttgggcattgtgtctgaactgcagctcaaactgaactccagcggctcctcagaagactcctttgttgaaattaggatggctgaaggagaagcagaaggttcagtaaaagaaatcaagcatagtcctgggcccacaagaacagtctccattggcacgagcagatctgcagagggggccaagaattacttggaacatgaggagttaactgtgagccagctcctgctgtgcctaagagaagggaatcagaaggtggagagacttgaaattgcactcaaggaggccaaagaaagagtttcagattttgaaaagaaagcaagtaatcgctctgagattgaaacccagacagaggggagcacagagaaagagaacgaggaagagaaaggcccagagactgttggaagcgaagtggaagcattgaaccttcaggtgacatctctgtttaaggagcttcaagaggctcatacaaaactcagtgaagccgagctaatgaagaagagacttcaagaaaagtgtcaggcccttgaaaggaaaaattctgcaactccatcagagttgaatgaaaagcaagagcttgtttatactaacaaaaagttagagctacaagtggaaagcatgctctcggaaatcaaaatggaacaggctaaaacggaggatgaaaagtccaaattagccatgttacagttgacacacaacaagcttcttcaggaacataatcatgcattgaaaacgattgaggagctaacaagaaaagagtcagaaaaagtggacagggcagtgctaaaggaactgagtgaaaaactggaactggcagagaaggctctggcttccaaacagctgcaaatggatgagatgaagcaaactattgccaagcaggaggaggacctggaaaccatgaccgtcctcagggctcagatggaagtttactgttctgattttcatgctgaaagagcagcaagagagaaaattcatgaggaaaaggagcaactggcattgcagctggcagttttgctgaaagagaatgatgctttcgaagatggaggcaggcagtccttgatggagatgcagagtcgtcatggggcgagaacaagtgaccccgaccagcaggcttaccttgttcaaagaggaactgaggacagggactggcagcaacagcggaatattccgattcattcctgccccaagtgtggagaggttctgcctgacatcgacacgctacagattcacgtgatggactgcatcatttaggtgttgatgtgtcacctccccaaaactgttggtaaatgtcagattttttcctccaagagttgtccttctgtgttatttgtttttcactcaaatattttacctcattattattgttttaaa

SEQ ID NO: 12 - protein, 571 aa, Macaca mulatta
  ref <308>: DATABASE ACCESSION NUMBER: AY228373
  ref <309>: DATABASE ENTRY DATE: 2002-03-30
  ref <313>: RELEVANT RESIDUES: (1)..(571)
MSHQPLSCLTEKGDSPSESTGNGPPHLAHPNLDTFTPEELLQQMKELLTENHQLKEAMKLNNQAMKGRFEELSAWTEKQKEERQFFETQSKEAKERLMALSHENEKLKEELGKLKGKSERSSEDPTDDSRLPRAEAEQEKDQLRTQVTRLQAEKADLLGIVSELQLKLNSSGSSEDSFVEIRMAEGEAEGSVKEIKHSPGPTRTVSIGTSRSAEGAKNYLEHEELTVSQLLLCLREGNQKVERLEIALKEAKERVSDFEKKASNRSEIETQTEGSTEKENEEEKGPETVGSEVEALNLQVTSLFKELQEAHTKLSEAELMKKRLQEKCQALERKNSATPSELNEKQELVYTNKKLELQVESMLSEIKMEQAKTEDEKSKLAMLQLTHNKLLQEHNHALKTIEELTRKESEKVDRAVLKELSEKLELAEKALASKQLQMDEMKQTIAKQEEDLETMTVLRAQMEVYCSDFHAERAAREKIHEEKEQLALQLAVLLKENDAFEDGGRQSLMEMQSRHGARTSDPDQQAYLVQRGTEDRDWQQQRNIPIHSCPKCGEVLPDIDTLQIHVMDCII

SEQ ID NO: 13 - DNA, 2076 bp, Macaca mulatta
  ref <308>: DATABASE ACCESSION NUMBER: AY228374
  ref <309>: DATABASE ENTRY DATE: 2002-03-30
  ref <313>: RELEVANT RESIDUES: (1)..(2076)
atcccggtcgggagttctctccaggcggcacgatgccaaggaagcagtaaccctgagcgaagccaagccgggcggcaggtgtggctttgacacctggtggtgccacttcctggtcttggatgagctgtacgcctctgtaaacccagcttcctcacctataaacagctgcctggttcagcgttaatgaagattagtcagtgacaggcctggtgtgccgagtccacacatagaacttctgcaatgtcccatcaacctctcagctgcctcactgaaaagggggacagccccagtgaaagcacaggaaatggacccccccatctggcccacccaaacctggacacattcaccccggaggagctgctgcagcagatgaaagagctcctgaccgagaaccaccagctgaaagaagccatgaagctaaataaccaagccatgaaagggcggtttgaggagctttcggcctggacagagaaacagaaggaagaacgccagttttttgagacacagagcaaagaagccaaagagcgtctcatggccttgagtcacgagaatgagaaattgaaggaagagcttggaaaactgaaagggaaatcagaaaggtcatctgaggaccccactgatgactccaggcttcccagggcagaagcggagcaggaaaaggaccagctcaggacccaggtgacgaggctacaagcagagaaggcagacctgttgggcattgtgtctgaactgcagctcaaactgaactccagcggctcctcagaagactcctttgttgaaattaggatggctgaaggagaagcagaaggttcagtaaaagaaatcaagcatagtcctgggcccacaagaacagtctccattggcacgagcagatctgcagagggggccaagaattacttggaacatgaggagttaactgtgagccagctcctgctgtgcctaagagaagggaatcagaaggtggagagacttgaaattgcactcaaggaggccaaagaaagagtttcagattttgaaaagaaagcaagtaatcgctctgagattgaaacccagacagaggggagcacagagaaagagaacgaggaagagaaaggcccagagactgttggaagcgaagtggaagcattgaaccttcaggtgacatctctgtttaaggagcttcaagaggctcatacaaaactcagtgaagccgagctaatgaagaagagacttcaagaaaagtgtcaggcccttgaaaggaaaaattctgcaactccatcagagttgaatgaaaagcaagagcttgtttatactaacaaaaagttagagctacaagtggaaagcatgctctcggaaatcaaaatggaacaggctaaaacggaggatgaaaagtccaaattagccatgttacagttgacacacaacaagcttcttcaggaacataatcatgcattgaaaacgattgaggagctaacaagaaaagagtcagaaaaagtggacagggcagtgctaaaggaactgagtgaaaaactggaactggcagagaaggctctggcttccaaacagctgcaaatggatgagatgaagcaaactattgccaagcaggaggaggacctggaaaccatgaccgtcctcagggctcagatggaagtttactgttctgattttcatgctgaaagagcagcaagagagaaaattcatgaggaaaaggagcaactggcattgcagctggcagttttgctgaaagagaatgatgctttcgaagatggaggcaggcagtccttgatggagatgcagagtcgtcatggggcgagaacaagtgaccccgaccagcaggcttaccttgttcaaagaggaactgaggacagggactggcagcaacagcggaatattccgattcattcctgccccaagtgtggagaggttctgcctgacatcgacacgctacagattcacgtgatggactgcatcatttaggtgttgatgtgtcacctccccaaaactgttggtaaatgtcagattttttcctccaagagttgtccttctgtgttatttgtttttcactcaaatattttacctcattattattgttttaaa

SEQ ID NO: 14 - protein, 571 aa, Macaca mulatta; identical to SEQ ID NO: 12
  ref <308>: DATABASE ACCESSION NUMBER: AY228374
  ref <309>: DATABASE ENTRY DATE: 2002-03-30
  ref <313>: RELEVANT RESIDUES: (1)..(571)

SEQ ID NO: 15 - DNA, 1787 bp, Rattus norvegicus
  ref <308>: DATABASE ACCESSION NUMBER: NM_145081
  ref <309>: DATABASE ENTRY DATE: 2003-04-06
  ref <313>: RELEVANT RESIDUES: (1)..(1787)
tgatctctgtggccggacctgttaccatgtcccatcaacctctgagctgcctgactgagaagggggacagctcctgtgagaccccaggaaatggaccctccaatatggttcaccccaacctggacacattcactcctgaggagctgctgcagcaaatgaaagaactcctggtcgagaaccaccagctgaaagaagccatgaagctaaataatcaagctatgaaagggcgatttgaggagctttctgcctggacagagaggcagaaggaagaacgccagttgtttgagatacaaagcaaagaggctaaggagcgcctcaaggccctgagtcatgaaaatgagaggctgaaggaagagcttggaaaactaaaagagaaatcagaaaggccatttgaagacatcacaggtagatgcgggtttcccagaaccgacttggagcaggaagtgggagcaactgaagaggcaggtggagcaggaagtggagcatctgaagatccaggtgaggcgccttcaggcttgagaaagcggaccttgctggggcattcgtctcagactgcagctcaagctcaactccggcggctcctcagaagactccttcgtggagacaggatgactgaaggagaggcggaaggggcaatgaaggagatgaggaacagcgctggacccacaaggacagactccatcatcatgggcaaatgtacagaggacgccaggacttgtgtggagtttgaggaactgactgtgagccaactcctgctttgcctcagggaaggaaaccaaaaggtggagagactcgagatcgcgctcagagaagccaaagaaagaatttcagattttgaaaagaaagcaaacggccattctgcgattgaaacccagacagaggggagcacacaaaaagaagaggaggacaaagacccagagagtgtgggaatcgaagtggaaactctgaacgttcaagtggcctctctgtttaaggggcttcaagaggcgcacacaaagctcagtgaggccgagctgatgaagaagagacttcaagaaaagtgtcaggctctggaaaggaagaactctgcaaccccatcggagctgaatgaaaagcaagagctcgtttacagtaactggaagttagagctgcaggtggagagcatgcgctcggaaatcaagatggagcaggccaagacagaggaggagaaatccaggttagccactcttcaggcaacacacgacaagctccttcaggaacacaataaagctctgagaacaattgaagaactaaccaaacaacaggcagaaaaagtggacaaggtgcagctgcaggagctcagcgagaagctggagctggcggagcaggctctggcgtccaagcagctccagatggatgagatgaagcagaccatcgccaagcaggaggaggacctggagaccatggccgtcctcagggctcagatggaggtgtactgttcagatttccatgctgagagggcagcaagggagaagatccatgaggaaaaggagcagctggccttgcagctcgccattttgctgaaagagaacaatgactttgaagatggaggcagtaggcagtccttgatggaaatgcagtgccggcacggggcgagaaccagcgactctgaccagcaggcttacctgtttcaaagaggagccaaggacatgagctggcagcatgggcagcagccccggagtattcccattcactcgtgccccaagtgtggggaggtcctgccggacattgacacgcttcagatacatgtgatggactgtatcatctga

SEQ ID NO: 16 - protein, 586 aa, Rattus norvegicus
  ref <308>: DATABASE ACCESSION NUMBER: NM_145081
  ref <309>: DATABASE ENTRY DATE: 2003-04-06
  ref <313>: RELEVANT RESIDUES: (1)..(586)
MSHQPLSCLTEKGDSSCETPGNGPSNMVHPNLDTFTPEELLQQMKELLVENHQLKEAMKLNNQAMKGRFEELSAWTERQKEERQLFEIQSKEAKERLKALSHENERLKEELGKLKEKSERPFEDITGRCGFPRTDLEQEVGATEEAGGAGSGASEDPGEAPSGLRKRTLLGHSSQTAAQAQLRRLLRRLLRGDRMTEGEAEGAMKEMRNSAGPTRTDSIIMGKCTEDARTCVEFEELTVSQLLLCLREGNQKVERLEIALREAKERISDFEKKANGHSAIETQTEGSTQKEEEDKDPESVGIEVETLNVQVASLFKGLQEAHTKLSEAELMKKRLQEKCQALERKNSATPSELNEKQELVYSNWKLELQVESMRSEIKMEQAKTEEEKSRLATLQATHDKLLQEHNKALRTIEELTKQQAEKVDKVQLQELSEKLELAEQALASKQLQMDEMKQTIAKQEEDLETMAVLRAQMEVYCSDFHAERAAREKIHEEKEQLALQLAILLKENNDFEDGGSRQSLMEMQCRHGARTSDSDQQAYLFQRGAKDMSWQHGQQPRSIPIHSCPKCGEVLPDIDTLQIHVMDCII

SEQ ID NO: 17 - DNA, 1799 bp, Sus scrofa
  ref <308>: DATABASE ACCESSION NUMBER: AF513722
  ref <309>: DATABASE ENTRY DATE: 2002-06-02
  ref <313>: RELEVANT RESIDUES: (1)..(1799)
atgtcccatcaacctctgagctgcctgactgagaagggggacagccccaccgaaaccacaggaaatggaccccccactctggctcacccaaaccttgacacgttcaccccacatgaactgctgcagcagatgagagagcttctaatcgagaaccatcagctgaaagaagccatgaagctaaataatcaagctatgaaagggcgatttgaggagctttcagcctggacagagaagcagaaggaagaacgccttttttttgagacccagagcaaagaagccaaagagcgcctaacggctctgagtcttgaaaatgaaaaactgaagcaagaacttggaaaactaaaagggaaaactgaaaggtcatttgaggacctcactggggaccccagggtccccaaggcggaagcagaacaggaagtagaacagctgaagacccaggtggcacgccttcaagctgaaaaggcggatctgctgggcatcgtgtctgaattgcagctcaagctgaactcaggtggcccctctgaagactcctttgttgaaatcaggatggctgagggagaagcagatgcagcaatgaaggaaatcaagacaagtcctgggcccataagaactgattccattgacacgagcaaatctgcagaaggtaccaggaattatttggaatttgaggaattaactgtgagccagctcctgctgtgtctaagggaaggaaaccagaaggtggagagacttgaaatcgccctcaaggaagccaaagaaagaattttagattttgaaaagaaagccaaggatcgttctgagactgagacccagacagaagagcacaaagaacaagagaaagaagaggagaaaagcccagaaactgttggaagtgaagtggaaatgttaaaccttcaggtgacaaccctgtttaaggagcttcaggaggctcacacgaaactcagtgaagctgagctcatgaagaagagacttcaagaaaaatgtcaggcacttgaaaggaaaaattctgcaaccccatcagagctgaatgaaaagcaagagcttctttataataacaaaaagttggagctccaagtggaaagcatgagatcagaaattaaaatggagcaagccaaaacagaagaggaaaagtccaaattaactactctacagttgacccacaacaggcttcttcaagaatacaataatgcactgaaaacaattgaggaactgaaaagaagagagtctgaaaaagtggataaggtggtgctgcaggaactgaatggaaagctggaaatggcagagaaggccctggcttccaagcagctccaaatggatgagatgaagcagaccattgccaagcaagagaaggacctggaaaccatggctgttctcagggctcagatggaggtatactgttctgactttcatgctgaaagagcagcaagagagaagattcatgaagaaaaggagcaactggcattgcagctggcagttttgctgaaagacgacaatgcttttgaagagggagccagcaggcaatccttgatggaaatgcagagccgtcatggggcaagagcaagtgatgctgaccagcaggcttttcttgttcaaagaggagctgaggatagaaactggctgcaacagcaacaacagaatattccaattcattcttgccccaaatgtggagaagttctgcctgacatagatacactactgattcacgttacggactgcatcatttaagtgctgacattttacttccccaaactgttggtaaatgtcagatttttttccccccaaaaaaaaaaaaaaaaaaa

SEQ ID NO: 18 - protein, 574 aa, Sus scrofa
  ref <308>: DATABASE ACCESSION NUMBER: AF513722
  ref <309>: DATABASE ENTRY DATE: 2002-06-02
  ref <313>: RELEVANT RESIDUES: (1)..(574)
MSHQPLSCLTEKGDSPTETTGNGPPTLAHPNLDTFTPHELLQQMRELLIENHQLKEAMKLNNQAMKGRFEELSAWTEKQKEERLFFETQSKEAKERLTALSLENEKLKQELGKLKGKTERSFEDLTGDPRVPKAEAEQEVEQLKTQVARLQAEKADLLGIVSELQLKLNSGGPSEDSFVEIRMAEGEADAAMKEIKTSPGPIRTDSIDTSKSAEGTRNYLEFEELTVSQLLLCLREGNQKVERLEIALKEAKERILDFEKKAKDRSETETQTEEHKEQEKEEEKSPETVGSEVEMLNLQVTTLFKELQEAHTKLSEAELMKKRLQEKCQALERKNSATPSELNEKQELLYNNKKLELQVESMRSEIKMEQAKTEEEKSKLTTLQLTHNRLLQEYNNALKTIEELKRRESEKVDKVVLQELNGKLEMAEKALASKQLQMDEMKQTIAKQEKDLETMAVLRAQMEVYCSDFHAERAAREKIHEEKEQLALQLAVLLKDDNAFEEGASRQSLMEMQSRHGARASDADQQAFLVQRGAEDRNWLQQQQQNIPIHSCPKCGEVLPDIDTLLIHVTDCII

(19) United States
(12) Patent Application Publication
(10) Pub. No.: US 2004/0191798 A1
(43) Pub. Date: Sep. 30, 2004

(54) Optineurin and glaucoma

(76) Inventors: Mansoor Sarfarazi, Farmington, CT (US); Tayebeh Rezaie, New Britain, CT (US); Anne Hawthorne Child, Keston (GB)

Correspondence Address: CANTOR COLBURN, LLP, 55 GRIFFIN ROAD SOUTH, BLOOMFIELD, CT, 06002

(21) Appl. No.: 10/609,133

(22) Filed: Jun. 26, 2003

Related U.S. Application Data
(63) Continuation of application No. 10/281,457, filed on Oct. 25, 2002, now abandoned.
Application No. 10/281,457 is a continuation of application No. 10/090,118, filed on Feb. 28, 2002, now abandoned.
Application No. 10/090,118 is a continuation-in-part of application No. 10/060,982, filed on Jan. 30, 2002.
(60) Provisional application No. 60/344,754, filed on Dec. 24, 2001.

(51) Int. Cl. C12Q 1/68; C07H 21/04; C07K 14/47

(52) U.S. Cl. 435/6; 435/69.1; 435/320.1; 435/325; 530/395; 536/23.5

(57) ABSTRACT

Methods of detection, prognosis and diagnosis of the presence or absence of optineurin-associated glaucoma or of an optineurin-associated increased risk of glaucoma are described, in which a sample is tested for the presence of certain sequence alterations in the gene, optineurin, that are associated with glaucoma or with an increased risk of glaucoma, as are methods in which a sample is tested for the presence of alterations in expression of the optineurin polypeptide, as well as methods in which a sample is tested for the presence of alterations in optineurin polypeptide expression or activity. Also described are methods of therapy of glaucoma, utilizing optineurin therapeutic agents.

### RELATED APPLICATIONS

This application is a continuation-in-part of U.S. Ser. No. 10/281,457, filed Oct. 25, 2002, which is a continuation of U.S. Ser. No. 10/090,118, filed Feb. 28, 2002, which is a continuation-in-part of U.S. Ser. No. 10/060,981, filed Jan. 30, 2002, which claims the benefit of U.S. Provisional Application No. 60/344,754, filed on Dec. 24, 2001, the contents of all of which are incorporated herein by reference in their entirety.

### GOVERNMENT SUPPORT

The invention was supported, in whole or in part, by grant RO1-EY09947 from the National Institutes of Health (National Eye Institute). The United States Government has certain rights in the invention.

### BACKGROUND OF THE INVENTION

Glaucoma is a progressive optic neuropathy characterized by a particular pattern of visual field loss and optic nerve head damage. Approximately 2.47 million people in the United States are affected with glaucoma (Quigley, H. A. and Vitale, S., Invest. Ophthalmol. Vis. Sci. 38:83 (1997)) and over 100,000 Americans are expected to develop this condition every year. Furthermore, over 67 million people worldwide are estimated to suffer from glaucoma (Quigley, H. A., Br. J. Ophthalmol. 80:389 (1996)). The most common form of this condition is primary open-angle glaucoma (POAG). Glaucomatous optic nerve damage and characteristic visual field loss are the two major clinical signs of this condition (Crick, R. P., Lancet 1:205 (1974); Quigley, H. A., N. Engl. J. Med. 328:1097 (1993); Wilson, R. and Matrone, J. in The Glaucomas, Vol. 2 pp. 753-768 (Ritch, S. M. and Krupin, T., Ed., St. Louis: Mosby, 1996)). Elevated intraocular pressure (IOP) is the most common known risk factor for glaucomatous damage, but it is not equivalent to the disease itself and numerous other risk factors are presently under investigation. Approximately one third to one half of patients with POAG (i.e., up to 1.2 million people in the United States alone) consistently have IOP within the statistically normal range of less than 22 mmHg (Tielsch, J. M. et al., JAMA 266:269 (1991); Hitchings, R. A., Br. J. Ophthalmol. 76:494 (1992); Grosskreutz, C. and Netland, P. A., Int. Ophthalmol. Clin. 34:173 (1994); Werner, E. B. in The Glaucomas, Vol. 2 pp. 768-797 (Ritch, S. M. and Krupin, T., Ed., St. Louis: Mosby, 1996). These patients have been considered to have low- or normal-tension glaucoma (LTG or NTG) and exhibit typical glaucomatous cupping of the optic nerve head and visual field loss (Hitchings, R. A. and Anderton, S. A., Br. J. Ophthalmol. 67:818 (1983)).

During the last decade, eight different genetic loci have been identified for different inherited forms of glaucoma. Three loci have been reported for primary congenital glaucoma (PCG), one for juvenile-onset (JOAG) and another five for adult-onset POAG (Sarfarazi, M. and Stoilov, I., in Ophthalmic Fundamentals: Glaucoma (Sassani, J. W., Ed. (Slack Inc., Thorofare, N. J. 1999), pp. 15-31). However, the causative gene has only been identified for two rare types of this condition, PCG (Stoilov, I. et al., Hum. Mol. Genet. 6:641 (1997)) and JOAG (Stone, E. M. et al., Science 275:668 (1997)). While ongoing studies show that cytochrome P4501B1 is the major gene for PCG (i.e., 85% of familial and 33% of sporadic cases) (Stoilov, I. et al., Am. J. Hum. Genet. 62:573 (1998)), mutations in the myocilin gene are primarily involved in a small subset of both JOAG and POAG subjects (i.e., 3.0-4.0%). Most of myocilin mutations are identified in JOAG cases (i.e., 2.0-2.5%), though there are other JOAG families that do not have a mutation in this gene (Stoilova, D. et al., J. Med. Genet. 35:989 (1998)). Furthermore, only a handful of mutations are reported in adult-onset POAG cases (i.e., 1.0-1.5%). As yet, no other gene has been identified that is responsible for the adult-onset POAG phenotype.

### SUMMARY OF THE INVENTION

The invention as described herein relates to mutations and other alterations associated with a gene originally identified in several British and other families, optineurin (OPTN), which has been associated with glaucoma and with increased risk of glaucoma. The invention further relates to methods of detecting, e.g., screening, prognosis or diagnosis, the presence or absence of glaucoma or of an increased risk of glaucoma and comprises, for example, the following embodiments:

An isolated nucleic acid molecule comprises SEQ ID NO: 1, an oligonucleotide of about 10 to about 50 nucleotides of SEQ ID NO: 1, SEQ ID NO: 3, an oligonucleotide of about 10 to about 50 nucleotides of SEQ ID NO: 3, SEQ ID NO: 5, an oligonucleotide of about 10 to about 50 nucleotides of SEQ ID NO: 5, or a complement of one of the foregoing nucleic acid molecules, wherein the nucleic acid molecule has an alteration in at least one nucleotide, and wherein the alteration is indicative of the presence of an optineurin-associated glaucoma or of an optineurin-associated risk of glaucoma.

In another embodiment, a purified polypeptide comprises SEQ ID NO: 2, a peptide of about 10 to about 50 contiguous amino acids of SEQ ID NO: 2, SEQ ID NO: 4, a peptide of about 10 to about 50 contiguous amino acids of SEQ ID NO: 4, SEQ ID NO: 6, or a peptide of about 10 to about 50 contiguous amino acids of SEQ ID NO: 6, wherein the polypeptide has an alteration in at least one amino acid, and wherein the alteration is indicative of the presence of an optineurin-associated glaucoma or of an optineurin-associated risk of glaucoma.

In another embodiment, an array of nucleic acid molecules attached to a solid support comprises an oligonucleotide comprising about 10 to about 50 nucleotides of SEQ ID NO: 1, about 10 to about 50 nucleotides of SEQ ID NO: 3, about 10 to about 50 nucleotides of SEQ ID NO: 5, or a complement of one of the foregoing oligonucleotides, wherein the oligonucleotide has an alteration in at least one nucleotide, and wherein the alteration is indicative of the presence of an optineurin-associated glaucoma or of an optineurin-associated risk of glaucoma.

In a further embodiment, a method of detecting (e.g., screening, prognosis or diagnosis) the presence or absence of an optineurin-associated glaucoma or an optineurin-associated risk of glaucoma in a sample from an individual comprises assessing the sample for an alteration in an optineurin nucleic acid; or an alteration in an optineurin polypeptide; wherein the alteration in the optineurin nucleic acid or the alteration in the optineurin polypeptide is indicative of the presence or the absence of an optineurin-associated glaucoma or an optineurin-associated risk of glaucoma.

A computer readable medium comprises an optineurin nucleic acid sequence, an optineurin polypeptide sequence, or a combination thereof. The optineurin nucleic acid sequence comprises SEQ ID NO: 1, an oligonucleotide of about 10 to about 50 nucleotides of SEQ ID NO: 1, SEQ ID NO: 3, an oligonucleotide of about 10 to about 50 nucleotides of SEQ ID NO: 3, SEQ ID NO: 5, an oligonucleotide of about 10 to about 50 nucleotides of SEQ ID NO: 5, or the complement of one of the foregoing nucleic acid sequences, wherein the nucleic acid sequence has an alteration in at least one nucleotide, and wherein the alteration is indicative of the presence or absence of optineurin-associated glaucoma or of an optineurin-associated risk of glaucoma. The optineurin polypeptide sequence comprises SEQ ID NO: 2, a peptide of about 10 to about 50 contiguous amino acids of SEQ ID NO: 2, SEQ ID NO: 4, a peptide of about 10 to about 50 contiguous amino acids of SEQ ID NO: 4, SEQ ID NO: 6, or a peptide of about 10 to about 50 contiguous amino acids of SEQ ID NO: 6, wherein the polypeptide sequence has an alteration in at least one amino acid, and wherein the alteration is indicative of the presence or absence of optineurin-associated glaucoma or of an optineurin-associated risk of glaucoma.

### BRIEF DESCRIPTION OF THE DRAWINGS

FIG. 1 depicts the genomic structure of optineurin, including approximate regions interacting with other known proteins, putative functional domains, sizes of exons, and position and types of mutations observed.

FIG. 2 depicts possible interactions of optineurin with other proteins and its potential involvement in alternative pathways of FAS-Ligand (left) and TNF-α (right). Interactions are depicted with solid arrows; downstream effects are depicted with open arrows; and a blocking effect of one protein on another is depicted with arrows ending in a circle.

The foregoing and other objects, features and advantages of the invention will be apparent from the following more particular description of preferred embodiments of the invention, as illustrated in the accompanying drawings.

### DETAILED DESCRIPTION OF THE INVENTION

The present invention relates to a gene identified as being associated with adult-onset, primary open angle glaucoma (POAG). As described herein, Applicant has identified a series of mutations in a gene, optineurin; the mutations are the principal cause of adult-onset low tension glaucoma (LTG)/primary open angle glaucoma (POAG) phenotype in their respective pedigrees. Applicant has additionally identified a mutation in the optineurin gene that is associated with an increased risk of glaucoma. Optineurin was originally identified as a tumor necrosis factor-α (TNF-α) inducible protein (Li, Y. et al., Mol. Cell. Biol. 18:1601 (1998)) and named FIP-2 (for adenovirus E3-14.7K interacting protein 2). Subsequently it was also identified as Huntingtin interacting protein L (HYPL) (Faber, P. W. et al., Hum. Mol. Genet. 7:1463 (1998)), NEMO-related protein (NRP) (Schwambom, K. et al., J. Biol. Chem. 275:22780 (2000)), transcription factor 111A interacting protein (TFIIIA-INTP) (Moreland, R. J. et al., Nucleic Acids Res. 28:1986 (2000)), and RAB8-interacting protein (Hattula, K. and Peranen, J., Curr. Bio. 10:1603 (2000)). This study utilizes “optineurin” for Optic Neuropathy Inducing protein as a new name for this protein. One or more of the mutations in optineurin may interfere with DNA or protein binding ability of optineurin, and another mutation leads to the premature truncation of the protein. Evidence indicates that interaction of optineurin with E3-14.7K protein probably utilizes TNF-α or Fas-Ligand pathways to mediate apoptosis, inflammation or vasoconstriction. Optineurin may also function through interactions with other proteins in cellular morphogenesis and membrane trafficking (RAB8), vesicle trafficking (Huntingtin), transcription activation (TFIIIA) and assembly or activity of two kinases.

Accordingly, the invention pertains to methods of therapy for glaucoma, and also methods and kits for diagnosing the presence or absence of glaucoma or of an increased risk of glaucoma in an individual, by detecting the presence or absence of alterations in the optineurin gene or the optineurin polypeptide, or by detecting an alteration in activity of the optineurin polypeptide or of an optineurin-interacting polypeptide. Glaucoma that is associated with the presence of one or more alterations in the optineurin gene or in the optineurin polypeptide is referred to herein as “optineurin-associated glaucoma”, and an increased risk of glaucoma associated with one or more alterations in the optineurin gene or in the optineurin polypeptide is referred to herein as “optineurin-associated increased risk of glaucoma”.

The term “glaucoma”, as used herein, icludes inheritable glaucomas, such as primary congenital or infantile glaucoma; primary open angle glaucoma (POAG), including both juvenile-onset and adult- or late-onset POAG; secondary glaucomas; pigmentary glaucoma; and low tension glaucoma (LTG) (also known as normal tension glaucoma (NTG)/normal pressure glaucoma (NPG)). In particular embodiments, the glaucoma can be primary open angle glaucoma (POAG) or the low tension subgroup of POAG. To the extent that the optineurin gene is implicated, other types of glaucoma are included in this definition including glaucomas associated with sporadic mutations. An “increased risk” of glaucoma, as used herein, refers to a likelihood of an individual for developing glaucoma, that is greater, by an amount that is statistically significant, than the likelihood of another individual or population of individuals for developing glaucoma. The methods of the invention can be used for detection, including screening, prognosis and diagnosis, of at-risk individuals and/or populations for glaucoma or the risk of glaucoma.

Methods of Detection, Including Prognosis and Diagnosis and Kits for Detection, Including Prognosis and Diagnosis

Optineurin Gene and Nucleic Acid-based Method

In one embodiment of the invention, detection (e.g., screening, prognosis or diagnosis) of optineurin-associated glaucoma, or of an optineurin-associated increased risk of glaucoma, can be made by detecting the presence or absence of an alteration in the optineurin gene that is associated with glaucoma or with an increased risk of glaucoma. As used herein, the term, “optineurin gene” refers to a nucleic acid (e.g., DNA, RNA, cDNA) encoding an optineurin polypeptide. A “gene” as used herein comprises not only translated nucleic acids, but also untranslated nucleic acids (e.g., promoter regions, 5′ untranslated regions, 3′ untranslated regions, introns, etc.).

For sequence information, see GenBank Accession # AF420371-3; SEQ ID NO: 1, 3, and 5 (nucleic acids encoding isoforms 1, 2 and 3 of optineurin, respectively); and SEQ ID NO: 2, 4 and 6 (polypeptides encoded by isoforms 1, 2 and 3 of optineurin, respectively). SEQ ID NO: 1 encodes isoform 1 of optineurin, including a 5′ untranslated region at nucleotides 1-310, the encoded isoform (SEQ ID NO: 2) at nucleotides 311 to 2044, and the 3′ untranslated region at nucleotides 2045-2077. SEQ ID NO: 3 encodes isoform 2 of optineurin, including a 5′ untranslated region at nucleotides 1-89, the encoded isoform (SEQ ID NO: 4) at nucleotides 90-1823, and the 3′ untranslated region at nucleotides 1824-1856. SEQ ID NO: 5 encodes isoform 3 of optineurin, including a 5′ untranslated region at nucleotides 1-241, the encoded isoform (SEQ ID NO: 6) at nucleotides 242-1975, and the 3′ untranslated region at nucleotides 1976-2008. These sequences are unaltered or wild type optineurin sequences.

Other nucleic acid sequences that have been described previously encode the protein now known as optineurin and are thus included in this disclosure. These sequences may differ at one or more nucleotides from SEQ ID NOs: 1, 3, and 5 due to sequencing errors or natural variations in the nucleic acid sequence. See also, for example, AH009711; AF061034; AF283519-27; see also, a Li, Y. et al., Mol. Cell. Biol. 18:1601 (1998); Faber, P. W. et al., Hum. Mol. Genet. 7:1463 (1998); Schwambom, K. et al., J. Biol. Chem. 275:22780 (2000); Moreland, R. J. et al., Nucleic Acids Res. 28:1986 (2000); and Hattula, K. and Peranen, J., Curr. Bio. 10:1603 (2000).

The term “isolated nucleic acid molecule” or “purified nucleic acid molecule” includes nucleic acid molecules that are separated from other nucleic acid molecules present in the natural source of the nucleic acid. For example, with regard to genomic DNA, the term “isolated” includes nucleic acid molecules that are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. By free of other cellular material, it is meant that an isolated nucleic acid molecule is greater than or equal to about 70%, 75%, 80%, 85%, 90%, 95% or 99% pure.

An “alteration” is a change (e.g., insertion, deletion, or change in one or more nucleotides) of the nucleic acid encoding optineurin polypeptide, as compared with the known sequence of nucleic acid encoding optineurin. The alteration can be a mutation in the optineurin gene, such as the insertion or deletion of a single nucleotide, or of more than one nucleotide, resulting in a frame shift mutation; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such mutation may be present in a single gene. Such sequence changes (e.g., exon shuffling) cause a mutation in the polypeptide encoded by the optineurin gene. For example, if the mutation is a frame shift mutation, the frame shift can result in a change in the encoded amino acids, and/or can result in the generation of a premature stop codon, causing generation of a truncated polypeptide. Alternatively, an alteration associated with glaucoma can be a sequence alteration that does not result in a change in the polypeptide encoded by the optineurin gene. Such an alteration may alter splicing sites, affect the stability or transport of mRNA, or otherwise affect the transcription or translation of the gene. A optineurin gene that has any of the mutations or sequence alterations described above is referred to herein as a “mutant gene”.

In specific embodiments of the invention, the alteration is a change from GAG to AAG at codon 50 of the optineurin gene; an insertion of AG in after codon 127; a change from CGG to CAG at codon 545; or a combination comprising one or more of the foregoing alterations. These alterations are associated with glaucoma, and the presence of one or more of these alterations is diagnostic for glaucoma. In another specific embodiment, the alteration is a change from ATG to AAG at codon 98; the presence of this alteration is indicative of an increased risk of glaucoma, and is diagnostic for an increased risk of glaucoma. Other alterations include a change from CCC to GCC at codon 16, a change from CAG to CAC at codon 42, a change from GAA to GTA at codon 92, a change from GAA to AAA at codon 322. Alterations include combinations of alterations such as combinations of alterations associated with glaucoma and those associated with an increased risk of glaucoma.

A method of detecting an indication (e.g., the presence or absence) of an optineurin-associated glaucoma or an optineurin-associated risk of glaucoma in a sample from an individual comprises assessing the sample for an alteration in an optineurin nucleic acid or an alteration in an optineurin polypeptide wherein the alteration is associated with the indication of an optineurin-associated glaucoma or an optineurin-associated risk of glaucoma. Detection of alterations in an optineurin nucleic acid or polypeptide can be used to screen individuals for an optineurin-associated glaucoma or an optineurin-associated risk of glaucoma.

In a first method of detetion of glaucoma or of an increased risk of glaucoma, hybridization methods, such as Southern analysis, can be used (see Current Protocols in Molecular Biology, Ausubel, F. et al., eds., John Wiley & Sons, including all supplements through 2001; this document is incorporated herein by reference in its entirety). For example, a test sample of genomic DNA, RNA, or cDNA, is obtained from an individual, such as, for example, an individual suspected of having, carrying a defect for, or being at increased risk for, glaucoma (the “test individual”). The individual can be an adult, child, or fetus. The test sample can be from a source which contains the nucleic acid (e.g., DNA, RNA), such as a blood sample, serum sample, lymph sample, sample of fluid from the eye (e.g., fluid from the anterior chamber), sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether an alteration in the optineurin gene is present.

If desired, amplification of the sample (e.g., by polymerase chain reaction) can be performed prior to assessment for the presence or absence of the alteration in the optineurin gene. Amplification can be used for all, or a portion of the nucleic acid comprising the optineurin gene so long as the portion contains the part of the optineurin gene that comprises the alteration (e.g., one or more exons, such as exon 4, exon 6, or other exons comprising an alteration, as described below). In a preferred embodiment, a portion contains at least one exon of the optineurin gene.

The presence or absence of the alteration can be indicated by hybridization of the gene in the genomic DNA, RNA, or cDNA to a nucleic acid probe. A “nucleic acid probe”, as used herein, is a single-stranded polynucleotide which hybridizes to the gene of interest (optineurin). The probe can comprise a portion of SEQ ID NO: 1, 3, or 5, preferably a contiguous portion, or a complement thereof. A nucleic acid probe can be an oligonucleotide of about 15 to about 30 nucleotides. Short probes generally require cooler temperatures to form sufficiently stable hybrid complexes with the template. The nucleic acid probe can be a DNA probe or an RNA probe; the nucleic acid probe contains at least one alteration in the optineurin gene. The probe can comprise the entire gene, a gene fragment, a vector comprising the gene, an exon of the gene, or a portion of nucleic acid which may be several hundered kilobases long, etc. Preferred nucleic acid probes are those comprising an alteration in the optineurin gene.

To detect, screen for, or diagnose the presence of glaucoma or of an increased risk of glaucoma, a hybridization sample can be formed by contacting the test sample containing an optineurin gene, with at least one nucleic acid probe. The hybridization sample is maintained under conditions which are sufficient to allow specific hybridization of the nucleic acid probe to the optineurin gene. “Specific hybridization”, as used herein, indicates exact hybridization (e.g., with no mismatches). Specific hybridization can be performed under high stringency conditions or moderate stringency conditions, for example.

“Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%, 98%). For example, certain high stringency conditions can be used which distinguish perfectly complementary nucleic acids from those of less complementarity.

“High stringency conditions”, “moderate stringency conditions” and “low stringency conditions” for nucleic acid hybridizations are explained in Current Protocols in Molecular Biology (Ausubel, F. M. et al., “Current Protocols in Molecular Biology”, John Wiley & Sons, (1998)). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology, 200:546-556 (1991). Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of 17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a prewarmed solution (42° C.) solution containing 0.2×SSC/0.1% SDS for 15 minutes at 42° C.; and a high stringency wash can comprise washing in prewarmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

In a particularly preferred embodiment, the hybridization conditions for specific hybridization are high stringency. Specific hybridization, if present, is then detected using standard methods. If specific hybridization occurs between the nucleic acid probe and the optineurin gene in the test sample, then the optineurin gene has the alteration that is present in the nucleic acid probe. More than one nucleic acid probe can also be used concurrently in this method. Specific hybridization of any one of the nucleic acid probes is indicative of the presence of an alteration in the optineurin gene that is associated with glaucoma or an increased risk of glaucoma, and is therefore diagnostic for optineurin-associated glaucoma or for an optineurin-associated increased risk of glaucoma. Absence of specific hybridization is indicative of the absence of an alteration in the optineurin gene that is associated with glaucoma or an increased risk of glaucoma, and is therefore diagnostic for the absence of optineurin-associated glaucoma or the absence of an optineurin-associated increased risk of glaucoma.

In another hybridization method, Northern analysis (see Current Protocols in Molecular Biology, Ausubel, F. et al., eds., John Wiley & Sons, supra) can be used to identify the presence or absence of an alteration associated with glaucoma or with an increased risk of glaucoma. For Northern analysis, a test sample of RNA is obtained from the individual by appropriate means. Specific hybridization of a nucleic acid probe, as described above, to RNA from the individual is indicative of the presence of an alteration in the optineurin gene that is associated with glaucoma or an increased risk of glaucoma, and is therefore diagnostic for optineurin-associated glaucoma or for an optineurin-associated increased risk of glaucoma. Absence of specific hybridization of a nucleic acid probe, as described above, to RNA from the individual is indicative of the absence of an alteration in the optineurin gene that is associated with glaucoma or an increased risk of glaucoma, and is therefore diagnostic for the absence of optineurin-associated glaucoma or of an optineurin-associate increased risk of glaucoma.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed to specifically hybridize to a gene having an alteration associated with glaucoma. Specific hybridization of a PNA probe, as described above, to RNA from the individual is indicative of the presence of an alteration in the optineurin gene that is associated with glaucoma or an increased risk of glaucoma, and is therefore diagnostic for optineurin-associated glaucoma or for an optineurin-associated increased risk of glaucoma. Absence of specific hybridization of a PNA probe, as described above, to RNA from the individual is indicative of the absence of an alteration in the optineurin gene that is associated with glaucoma or an increased risk of glaucoma, and is therefore diagnostic for the absence of optineurin-associated glaucoma or of an optineurin-associate increased risk of glaucoma.

In another method of the invention, mutation analysis by restriction digestion can be used to detect mutant genes, or genes containing alterations, if the mutation or alteration in the gene results in the creation or elimination of a restriction site. A test sample containing genomic DNA is obtained from the individual. Polymerase chain reaction (PCR) can be used to amplify the optineurin gene (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the mutation or alteration in the optineurin gene that is associated with glaucoma or an increased risk of glaucoma, and therefore is prognostic or diagnostic for the presence or absence of optineurin-associated glaucoma or optineurin-associated increased risk for glaucoma.

Sequence analysis can also be used to detect specific alterations in the optineurin gene. A test sample of DNA or RNA is obtained from the test individual. PCR or other appropriate methods can be used to amplify the gene, and/or its flanking sequences, if desired. The sequence of the optineurin gene, or a fragment of the gene (e.g., one or more exons), or cDNA, or fragment of the cDNA, or mRNA, or fragment of the mRNA, is determined, using standard methods. The sequence of the gene, gene fragment, cDNA, cDNA fragment, mRNA, or mRNA fragment is compared with the known nucleic acid sequence of the gene, cDNA, or mRNA, as appropriate. The presence of an alteration in the optineurin gene indicates that the individual has an alteration associated with glaucoma or with an increased risk of glaucoma, and is therefore prognostic or diagnostic for optineurin-associated glaucoma or for an optineurin-associated increased risk of glaucoma. The absence of an alteration in the optineurin gene indicates that the individual does not have an alteration associated with glaucoma or with an increased risk of glaucoma, and is therefore diagnostic for the absence of optineurin-associated glaucoma or of an optineurin-associated increased risk of glaucoma.

Allele-specific oligonucleotides can also be used to detect the presence of an alteration in the optineurin gene, through the use of dot-blot hybridization of amplified oligonucleotides with allele-specific oligonucleotide (ASO) probes (see, for example, Saiki, R. et al., (1986), Nature (London) 324:163-166). An “allele-specific oligonucleotide” (also referred to herein as an “allele-specific oligonucleotide probe”) is an oligonucleotide of approximately 10-50 bases, preferably approximately 15-30 bases, preferably contiguous bases, that specifically hybridizes to the optineurin gene, and that contains an alteration associated with glaucoma or with increased risk of glaucoma. An allele-specific oligonucleotide probe that is specific for particular alterations in the optineurin gene can be prepared, using standard methods (see Current Protocols in Molecular Biology, supra). To identify alterations in the gene that are associated with glaucoma or with an increased risk of glaucoma, a test sample of DNA is obtained from the individual. PCR can be used to amplify all or a fragment of the optineurin gene, and its flanking sequences. The DNA containing the amplified optineurin gene (or fragment of the gene) is dot-blotted, using standard methods (see Current Protocols in Molecular Biology, supra), and the blot is contacted with the oligonucleotide probe. The presence of specific hybridization of the probe to the amplified optineurin gene is then detected. Specific hybridization of an allele-specific oligonucleotide probe to DNA from the individual is indicative of an alteration in the optineurin gene that is associated with glaucoma or an increased risk of glaucoma, and is therefore indicative of optineurin-associated glaucoma or of an optineurin-associated increased risk of glaucoma. An absence of specific hybridization of an allele-specific oligonucleotide probe to DNA from the individual is indicative of an absence of an alteration in the optineurin gene that is associated with glaucoma or an increased risk of glaucoma, and is therefore indicative of an absence of optineurin-associated glaucoma or of an optineurin-associated increased risk of glaucoma.

Other methods of nucleic acid analysis can be used to detect alterations in the optineurin gene. Representative methods include direct manual sequencing (Church and Gilbert, (1988), Proc. Natl. Acad. Sci. USA 81:1991-1995; Sanger, F. et al. (1977) Proc. Natl. Acad. Sci. 74:5463-5467; Beavis et al. U.S. Pat. No. 5,288,644); automated fluorescent sequencing; single-stranded conformation alteration assays (SSCP); clamped denaturing gel electrophoresis (CDGE); denaturing gradient gel electrophoresis (DGGE) (Sheffield, V. C. et al. (1989) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (CMC) (Cotton et al. (1985) Proc. Natl. Acad. Sci. USA 85:4397-4401); RNAse protection assays (Myers, R. M. et al. (1985) Science 230:1242); use of polypeptides which recognize nucleotide mismatches, such as E. coli mutS protein; allele-specific PCR, for example.

Optineurin Polypeptide-Based Methods

This disclosure also relates to optineurin polypeptides, such as SEQ ID NOs: 2, 4 and 6. Preferred optineurin polypeptides are those containing alterations in their amino acid sequences, and wherein the alterations are associated with the presence of optineurin-associated glaucoma or of an optineurin-associated risk of glaucoma in an individual. In addition, also provided are polypeptides corresponding to a portion of SEQ ID NO: 2, SEQ ID NO: 4 and/or SEQ ID NO: 6 referred to as polypeptide fragments. A polypeptide fragment corresponds to about 10 to about 50 contiguous amino acids of SEQ ID NO: 2, SEQ ID NO: 4 or SEQ ID NO: 6. Within this range, greater than or equal to about 15 contiguous amino acids is preferred, with greater than or equal to about 20 amino acids more preferred. Also within this range, less than or equal to about 30 amino acids is preferred, with less than or equal to about 25 amino acids more preferred. Preferably, the polypeptide fragment is an immunogenic fragment. As used herein, “immunogenic fragment” refers to a polypeptide fragment that can directly/indirectly induce a specific immune response in appropriate animals or cells and bind with specific antibodies.

An “isolated” or “purified” polypeptide, immunogenic fragment, or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of polypeptide in which the polypeptide is separated from cellular components of the cells from which it is isolated or recombinantly produced. Thus, polypeptide that is substantially free of cellular material includes preparations of polypeptide having less than about 30%, 20%, 10%, or 5% (by dry weight) of heterologous protein (also referred to herein as a “contaminating protein”). When the polypeptide or biologically active portion thereof is recombinantly produced, it is also preferably substantially free of culture medium, i.e., culture medium represents less than about 20%, 10%, or 5% of the volume of the polypeptide preparation. When the polypeptide is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

In another embodiment of the invention, diagnosis of the presence or absence of optineurin-associated glaucoma or of an optineurin-associated increased risk of glaucoma can also be made by examining expression and/or composition of the optineurin polypeptide. A test sample from an individual can be assessed for the presence or absence of an alteration in the expression and/or an alteration in composition of the polypeptide encoded by the optineurin gene. An alteration in expression of a polypeptide encoded by an optineurin gene can be, for example, an alteration in the quantitative polypeptide expression (i.e., the amount of polypeptide produced); an alteration in the composition of a polypeptide encoded by an optineurin gene is an alteration in the qualitative polypeptide expression. Both such alterations can also be present. An “alteration” in the polypeptide expression or composition, as used herein, refers to an alteration in expression or composition in a test sample, as compared with the expression or composition of polypeptide by an optineurin gene in a control sample. A control sample is a sample that corresponds to the test sample (e.g., is from the same type of cells), and is from an individual who is not affected by glaucoma and who is not at increased risk for glaucoma. An alteration in the expression or composition of the polypeptide in the test sample, as compared with the control sample, is indicative of optineurin-associated glaucoma or of an increased risk of optineurin-associated glaucoma. Absence of an alteration in the expression or composition of the polypeptide in the test sample, as compared with the control sample, is indicative of an absence of optineurin-associated glaucoma or of an increased risk of optineurin-associated glaucoma. Polypeptide alterations include, for example, a glutamic acid to lysine at codon 50, a premature stop after codon 127, an arginine to glutamine at codon 545, a methionine to lysine at codon 98, or a combination comprising one or more of the foregoing alterations.

Various means of examining expression or composition of the polypeptide encoded by the optineurin gene can be used, including spectroscopy, colorimetry, electrophoresis, isoelectric focussing, and immunoassays (e.g., David et al., U.S. Pat. No. 4,376,110) such as immunoblotting (see also Current Protocols in Molecular Biology, particularly chapter 10). For example, Western blotting analysis, using an antibody that specifically binds to a polypeptide encoded by a mutant optineurin gene, or an antibody that specifically binds to a polypeptide encoded by a non-mutant gene, can be used to identify the presence in a test sample of a polypeptide encoded by a polymorphic or mutant optineurin gene, or the absence in a test sample of a polypeptide encoded by a non-polymorphic or non-mutant gene. The presence of a polypeptide encoded by a polymorphic or mutant gene, or the absence of a polypeptide encoded by a non-polymorphic or non-mutant gene, is indicative of an alteration associated with glaucoma or an increased risk of glaucoma, and is therefore prognostic or diagnostic for optineurin-associated glaucoma or for an optineurin-associated increased risk of glaucoma. The absence of a polypeptide encoded by a polymorphic or mutant gene, or the presence of a polypeptide encoded by a non-polymorphic or non-mutant gene, is indicative of the absence of an alteration associated with glaucoma or an increased risk of glaucoma, and is therefore prognostic or diagnostic for an absence of optineurin-associated glaucoma or for an optineurin-associated increased risk of glaucoma.

In one embodiment of this method, the level or amount of polypeptide encoded by an optineurin gene in a test sample is compared with the level or amount of the polypeptide encoded by the optineurin gene in a control sample. A level or amount of the polypeptide in the test sample that is higher or lower than the level or amount of the polypeptide in the control sample, such that the difference is statistically significant, is indicative of an alteration in the expression of the polypeptide encoded by the optineurin gene, and is diagnostic for optineurin-associated glaucoma or for an optineurin-associated increased risk of glaucoma. A level or amount of the polypeptide in the test sample that is not statistically different from the level or amount of the polypeptide in the control sample, is indicative of the absence of an alteration in the expression of the polypeptide encoded by the optineurin gene, and is diagnostic for an absence of optineurin-associated glaucoma or of an optineurin-associated increased risk of glaucoma.

Alternatively, the composition of the polypeptide encoded by an optineurin gene in a test sample is compared with the composition of the polypeptide encoded by the optineurin gene in a control sample. A difference in the composition of the polypeptide in the test sample, as compared with the composition of the polypeptide in the control sample, is diagnostic for optineurin-associated glaucoma or for an optineurin-associated increased risk of glaucoma. An absence of difference in the composition of the polypeptide in the test sample, as compared with the composition of the polypeptide in the control sample, is diagnostic for an absence of optineurin-associated glaucoma or of an optineurin-associated increased risk of glaucoma.

In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of optineurin-associated glaucoma or an optineurin-associated increased risk of glaucoma. Absence of both a difference in the amount or level, and a difference in the composition, is indicative of an absence of optineurin-associated glaucoma or an optineurin-associated increased risk of glaucoma.

Other Optineurin Polypeptide-based Methods

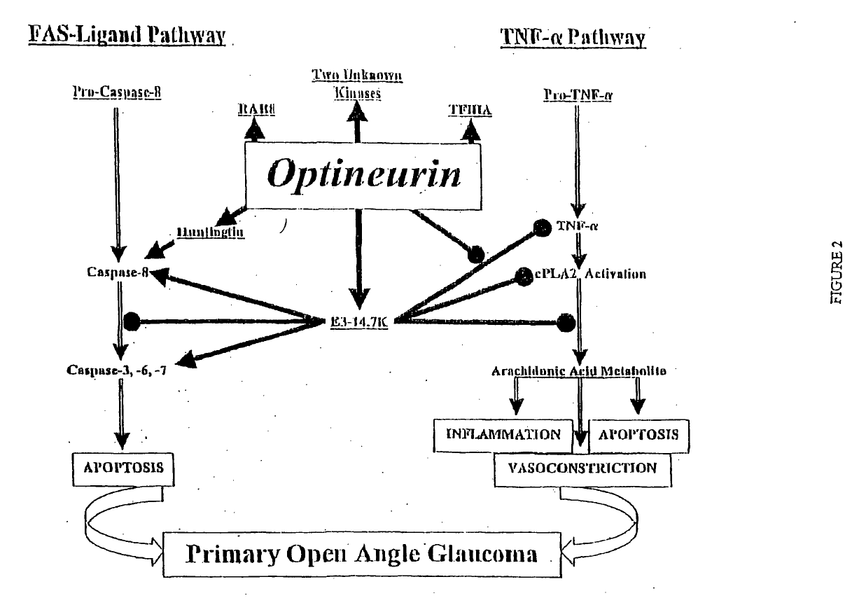

In another embodiment of the invention, detection (e.g., screening, prognosis or diagnosis) of the presence or absence of optineurin-associated glaucoma or of an optineurin-associated increased risk of glaucoma can also be made by examining activity of the optineurin polypeptide. A test sample from an individual is assessed for the presence or absence of an alteration in the activity of the polypeptide encoded by the optineurin gene, as compared with the activity of the polypeptide encoded by the optineurin gene in a control sample. As described below and as shown in FIG. 2, optineurin may interact with a variety of proteins, including E3-14.7K, components of the TNF-α pathway, and components of the FAS-ligand pathway. These proteins are referred to herein as “optineurin-interacting polypeptides”.

An alteration in activity of a polypeptide encoded by an optineurin gene can be, for example, an increase or decrease of interaction between optineurin polypeptide and an optineurin-interacting polypeptide. The level or amount of optineurin interaction with an optineurin-interacting polypeptide can be assessed in the test sample and in a control sample (for example, a sample comprising native optineurin polypeptide). A difference in the amount or level of interaction in the test sample, compared to the control sample, is indicative of the presence of an alteration in optineurin, and is thereby indicative of optineurin-associated glaucoma or an optineurin-associated increased risk of glaucoma. Absence of a difference in the amount or level of interaction, is indicative of the absence of such an alteration and thereby indicative of the absence of optineurin-associated glaucoma or an optineurin-associated increased risk of glaucoma. For example, it is possible that the C-terminal part of optineurin interacts with Huntingtin. An alteration in a test sample, of the amount or level of interaction between optineurin and Huntingtin, as compared with the amount or level of interaction between optineurin and Huntingtin in a control sample, can be indicative of optineurin-associated glaucoma or an optineurin-associated increased risk of glaucoma.

In another example, the amount or level of activity of an optineurin-interacting polypeptide can be used as an indirect measure of the amount or level of activity of optineurin. A difference in the amount or level of activity of the optineurin-interacting polypeptide in the test sample, compared to the control sample, is indicative of the presence of an alteration in optineurin, and is thereby indicative of optineurin-associated glaucoma or an optineurin-associated increased risk of glaucoma. Absence of a difference in the amount or level of activity of the optineurin-interacting polypeptide, is indicative of the absence of such an alteration and thereby indicative of the absence of optineurin-associated glaucoma or an optineurin-associated increased risk of glaucoma. For example, it is possible that optineurin (either directly or though its interaction with other proteins) can affect the levels of a protein that can be assessed and be used as a proxy for the level of optineurin activity. An alteration in a test sample of the amount or level of an interacting protein (e.g., an increased amount of the protein), as compared with the amount or level of the protein in a control sample, is indicative of the presence of a mutation in optineurin and thereby indicative of the presence of optineurin-associated glaucoma or an optineurin-associated increased risk of glaucoma.

Kits

Kits useful in the methods of detection, screening, prognosis and/or diagnosis can comprise components useful the methods described herein, including for example, hybridization probes, restriction enzymes (e.g., for RFLP analysis), allele-specific oligonucleotides, antibodies which bind to mutant or to non-mutant (native) optineurin polypeptide, means for amplification of nucleic acids comprising the optineurin gene, or means for analyzing the nucleic acid sequence of the optineurin gene or for analyzing the amino acid sequence of the optineurin polypeptide, etc. A kit may also comprise a reagent suitable for performing a detection method such as a hybridization reaction, an immunological reaction, and the like.

Array-Based Methods

In another aspect, this disclosure includes an array that includes a substrate having a plurality of addresses. At least one address of the plurality includes a capture probe that binds specifically to an optineurin nucleic acid or polypeptide comprising an alteration. Preferred alterations are those that indicate the presence of optineurin-associated glaucoma or of an optineurin-associated risk of glaucoma in an individual. The array can have a density of about 10, 50, 100, 200, 500, 1,000, 2,000, or 10,000 or more addresses/cm², and ranges between. The plurality of addresses can include 10, 100, 500, 1,000, 5,000, 10,000, 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the first plurality can be disposed on the array.

At least one address of the plurality can include a nucleic acid capture probe that hybridizes specifically to an optineurin polynucleotide, including the sense and/or anti-sense strand. Preferably the nucleic acid capture probe comprises an optineurin alteration. A subset of addresses of the plurality of addresses can have a nucleic acid capture probe for optineurin. Each address of the subset can include a capture probe that hybridizes to a different region of an optineurin nucleic acid. Addresses of the subset include a capture probe for an optineurin nucleic acid. Each address of the subset can be unique, overlapping, and/or complementary to a different alteration of optineurin. The array can be used to detect or sequence optineurin polynucleotides by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

At least one address of the plurality can include a polypeptide capture probe that binds specifically to an optineurin polypeptide or fragment thereof, preferably an optineurin polypeptide comprising an optineurin alteration. The polypeptide can be a naturally-occurring interaction partner of the optineurin polypeptide. Preferably, the polypeptide is an antibody, e.g., an antibody such as a monoclonal antibody.

A method of analyzing the expression of optineurin includes providing an array as described above; contacting the array with a sample and detecting binding of an optineurin molecule (e.g., nucleic acid or polypeptide) to the array. The array can be a nucleic acid array. Optionally the method further includes amplifying nucleic acid from the sample prior to or during contact with the array.

The array can be used to assay gene expression in a tissue to ascertain tissue specificity of genes in the array, particularly the expression of optineurin. If a sufficient number of diverse samples are analyzed, clustering (e.g., hierarchical clustering, k-means clustering, Bayesian clustering, and the like) can be used to identify other genes which are co-regulated with optineurin. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

Cells can be contacted with a therapeutic agent. The expression profile of the cells can then be determined using the array, and the expression profile compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the assay can be used to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

The array can be used to monitor expression of one or more genes in the array with respect to time. For example, samples obtained from different time points can be probed with the array. Such analysis can identify and/or characterize the development of an optineurin-associated glaucoma. The method can also evaluate the treatment and/or progression of an optineurin-associated glaucoma.

The array is also useful for ascertaining differential expression patterns of one or more genes in normal and abnormal cells. This approach provides a battery of genes (e.g., including optineurin) that could serve as a molecular target for diagnosis or therapeutic intervention.

The array can be a polypeptide array. At least one address of the plurality has disposed thereon an optineurin polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. Each address of the plurality can have disposed thereon a polypeptide at least 60, 70, 80, 85, 90, 95 or 99% identical to an optineurin polypeptide or fragment thereof. For example, multiple variants of an optineurin polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect an optineurin binding compound, e.g., an antibody in a sample from a subject with specificity for an optineurin polypeptide or the presence of an optineurin-binding protein or ligand.

The array is also useful for ascertaining the effect of the expression of a gene on the expression of other genes in the same cell or in different cells (e.g., ascertaining the effect of optineurin expression on the expression of other genes). This provides, for example, for a selection of alternate molecular targets for therapeutic intervention if the ultimate or downstream target cannot be regulated.

In another aspect, a method of analyzing a plurality of probes is disclosed. The method is useful, e.g., for analyzing gene expression. The method includes use of a two dimensional array having a plurality of addresses, each address of the plurality being positionally distinguishable from each other address of the plurality having a unique capture probe. The array can be contacted with one or more inquiry probes (e.g., probes other than an optineurin nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

A method of analyzing a plurality of probes in a sample is described. The method is useful, e.g., for analyzing gene expression. The method includes use of a two dimensional array having a plurality of addresses, each address of the plurality being positionally distinguishable from each other address of the plurality having a unique capture probe. The array can be contacted with a first sample from a cell or subject which express or mis-express optineurin or from a cell or subject in which an optineurin-mediated response has been elicited. The array is then contacted with a second sample in which an optineurin mediated response has not been elicited, or has been elicited to a lesser extent than in the first sample. Binding of the first sample is compared with the binding of the second sample. Binding, e.g., in the case of a nucleic acid, hybridization with a capture probe at an address of the plurality, can be detected, e.g., by signal generated from a label attached to the nucleic acid, polypeptide, or antibody. The same array can be used for both samples or different arrays can be used. If different arrays are used, the plurality of addresses with capture probes should be present on both arrays.

Genetic mutations in optineurin can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of an optineurin nucleic acid or a putative variant (e.g., allelic variant) thereof. A probe can have one or more mismatches to a region of an optineurin nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in optineurin can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

Computer Readable Media

The optineurin nucleotide or amino acid sequences are also provided in a variety of mediums to facilitate use thereof. Medium refers to a manufacture, other than an isolated nucleic acid or polypeptide molecule, which contains an optineurin nucleotide or amino acid sequence. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exists in nature or in purified form. The manufacture comprises at least one optineurin nucleic acid or polypeptide sequence that comprises an optineurin alteration.

An optineurin nucleotide or amino acid sequence can be recorded on computer readable media. As used herein, “computer readable medium” refers to a medium that can be read and accessed directly by a computer. Such media include, but are not limited to: magnetic storage media, such as floppy discs, hard disc storage media, and magnetic tape; optical storage media such as CD-ROM; electrical storage media such as RAM and ROM; and hybrids of these categories such as magnetic/optical storage media. The skilled artisan will readily appreciate how the presently known computer readable media can be used to create a manufacture comprising a computer readable medium having recorded thereon a nucleotide or amino acid sequence of the present invention.

As used herein, “recorded” refers to a process for storing information on computer readable media. The skilled artisan can readily adopt the presently known methods for recording information on computer readable media to generate manufactures comprising optineurin nucleotide or amino acid sequence information.

A variety of data storage structures are available to a skilled artisan for creating a computer readable medium having recorded thereon an optineurin nucleotide or amino acid sequence. The choice of the data storage structure will generally be based on the means chosen to access the stored information. In addition, a variety of data processor programs and formats can be used to store the nucleotide sequence information on computer readable medium. The sequence information can be represented in a word processing text file, formatted in commercially-available software such as WordPerfect and MicroSoft Word, or represented in the form of an ASCII file, stored in a database application, such as DB2, Sybase, Oracle, or the like. The skilled artisan can readily adapt a number of data processor structuring formats (e.g., text file or database) in order to obtain a computer readable medium having recorded thereon the nucleotide sequence information of the present invention.

By providing optineurin nucleotide or amino acid sequences in computer readable form, the skilled artisan can routinely access the sequence information for a variety of purposes. For example, one skilled in the art can use the optineurin nucleotide or amino acid sequences in computer readable form to compare a target sequence or target structural motif with the sequence information stored within the data storage means. Search means are used to identify fragments or regions of the optineurin sequences which match a particular target sequence or target motif.

A target sequence can be a nucleotide or amino acid sequence of six or more nucleotides or two or more amino acids. A skilled artisan can readily recognize that the longer a target sequence is, the less likely a target sequence will be present as a random occurrence in the database. The most preferred sequence length of a target sequence is about 10 to about 100 amino acids or about 30 to about 300 nucleotide residues. However, it is well recognized that commercially important fragments, such as sequence fragments involved in gene expression and protein processing, may be of shorter length.

A target structural motif or target motif refers to a rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in computer-based systems. Examples of such software include, but are not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

Methods of Therapy

The present invention also pertains to methods of treatment (prophylactic and/or therapeutic) for glaucoma or for an increased risk of glaucoma, using an optineurin therapeutic agent. The methods can be used not only for individuals diagnosed with, or suspected of having, optineurin-associated glaucoma or an optineurin-associated increased risk of glaucoma; the methods can also be used for individuals diagnosed with or suspected of having glaucoma or an increased risk of glaucoma other than those associated with optineurin, as they may similarly be beneficial in such individuals by altering the course of the glaucoma. As described below and as shown in FIG. 2, optineurin may interact with a variety of proteins, including E3-14.7K, components of the TNF-α pathway, and components of the FAS-ligand pathway. These proteins, referred to as “optineurin-interacting polypeptides”, are appropriate targets for optineurin therapeutic agents, to alter the activity and interaction between them and optineurin and thereby treat glaucoma.

An “optineurin therapeutic agent” is an agent used for the treatment of glaucoma, that alters (e.g., enhances or inhibits) optineurin polypeptide activity and/or optineurin gene expression (e.g., an optineurin agonist or antagonist). The therapy is designed to inhibit, alter, replace or supplement activity of the optineurin polypeptide in an individual, or to inhibit, alter, replace or supplement activity of an optineurin-interacting polypeptide in an individual.

An optineurin therapeutic agent can alter optineurin activity or gene expression by a variety of means, such as, for example, by providing additional protein or by upregulating the transcription or translation of optineurin; by altering posttranslational processing of the optineurin polypeptide; by altering transcription of splicing variants of optineurin; or by altering optineurin polypeptide activity (e.g., by binding to optineurin), or by altering (upregulating or downregulating) the transcription or translation of optineurin. Other optineurin therapeutic agents can target optineurin-interacting polypeptides, to alter activity or expression of genes encoding optineurin-interacting polypeptides or of other genes in the pathways in which optineurin takes part.

Representative optineurin therapeutic agents include several different classes of agents.

Nucleic Acids

In one embodiment, the optineurin therapeutic agent can be a nucleic acid, such as a gene, cDNA, mRNA, a nucleic acid encoding an optineurin polypeptide (e.g., SEQ ID NO: 1, 3, or 5) or a variant of optineurin, wherein a nucleic acid encoding a variant (a variant nucleic acid molecule) is one that is not necessarily found in nature but which encodes a polypeptide having the amino acid sequence of optineurin. Thus, for example, DNA molecules which comprise a sequence that is different from the naturally-occurring nucleotide sequence but which, due to the degeneracy of the genetic code, encode optineurin, are contemplated, as are nucleotide sequences encoding portions (fragments), or encoding variant polypeptides such as analogues or derivatives of optineurin. Such variants can be naturally-occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions. Preferably the nucleotide (and/or resultant amino acid) changes are silent or conserved; that is, they do not alter the characteristics or activity of the mutant optineurin (e.g., the ability to interact with other specific proteins, as described in detail below). Other alterations of the nucleic acid molecules can include, for example, labelling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequences via hydrogen bonding and other chemical interactions. Such molecules include, for example, those in which peptide linkages substitute for phosphate linkages in the backbone of the molecule.

Other optineurin therapeutic agents include aptamers, which are DNA or RNA molecules that have been selected based on their ability to bind other molecules. (See, for example, the aptamer data base, at aptamer.icmb.utexas.edu/; see also, Gening, L. V. et al., Biotechniques 31(4):828, 830, 832, 834 (2001);

Proteins and Polypeptides

In another embodiment, the optineurin therapeutic agent can be optineurin polypeptide (e.g., SEQ ID NO: 2, 4 and 6), a peptidomimetic, a portion of an optineurin polypeptide, or a derivative of an optineurin polypeptide, or another splicing variant encoded by the optineurin gene or fragments or derivatives thereof. Fusion proteins or other polypeptides comprising fragments (particularly fragments retaining an activity of optineurin) can be used, as can optineurin polypeptides encompassing sequencing variants.

Active fragments perform one or more of the same functions as the whole optineurin polypeptide (the ability to interact with other specific proteins, as described below). For example, active fragments can comprise a domain, segment, or motif that has been identified by analysis of the protein sequence using well-known methods, e.g., signal peptides, extracellular domains, one or more transmembrane segments or loops, ligand binding regions, zinc finger domains, DNA binding domains, acylation sites, glycosylation sites, or phosphorylation sites. Active fragments can be discrete (not fused to other amino acids or polypeptides) or can be within a larger polypeptide. Further, several fragments can be comprised within a single larger polypeptide.

Variants include a substantially homologous polypeptide encoded by the same genetic locus in an organism, i.e., an allelic variant, as well as other splicing variants. Variants also encompass polypeptides derived from other genetic loci in an organism, but having significant homology to a polypeptide encoded by an optineurin gene or nucleic acid as described above. Variants also include proteins substantially homologous or identical to these proteins but derived from another organism, i.e., an ortholog. Variants also include proteins that are substantially homologous or identical to these proteins that are produced by chemical synthesis. Variants also include proteins that are substantially homologous or identical to these proteins and that are produced by recombinant methods. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990). A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region. Amino acids that are essential for function can be identified by methods known in the art, such as site-directed mutagenesis or alanine-scanning mutagenesis (Cunningham et al., Science, 244:1081-1085 (1989)). Sites that are critical for polypeptide activity can also be determined by structural analysis such as crystallization, nuclear magnetic resonance or photoaffinity labelling (Smith et al., J. Mol. Biol., 224:899-904 (1992); de Vos et al. Science, 255:306-312 (1992)).

The optineurin polypeptides and other polypeptides described herein can also be isolated from naturally-occurring sources, chemically synthesized or recombinantly produced. For example, a nucleic acid molecule described herein can be used to produce a recombinant form of the encoded protein via microbial or eukaryotic cellular processes. Ligating the polynucleotide sequence into a gene construct, such as an expression vector, and transforming or transfecting into hosts, either eukaryotic (yeast, avian, insect, plant or mammalian) or prokaryotic (bacterial cells), are standard procedures used in producing other well known proteins. Similar procedures, or modifications thereof, can be employed to prepare recombinant proteins by microbial means or tissue-culture technology. The proteins can be isolated or purified (e.g., to homogeneity) from cell culture by a variety of processes. These include, but are not limited to, anion or cation exchange chromatography, ethanol precipitation, affinity chromatography and high performance liquid chromatography (HPLC). The particular method used will depend upon the properties of the protein; appropriate methods will be readily apparent to those skilled in the art. For example, with respect to protein or protein identification, bands identified by gel analysis can be isolated and purified by HPLC, and the resulting purified protein can be sequenced. Alternatively, the purified protein can be enzymatically digested by methods known in the art to produce protein fragments which can be sequenced. The sequencing can be performed, for example, by the methods of Wilm et al. (Nature 379(6564):466-469 (1996)). The protein may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990).

Antibodies and Other Small Molecules

In another embodiment, the optineurin therapeutic agent can be an antibody (e.g., an antibody to a mutant optineurin polypeptide, an antibody to a non-mutant optineurin polypeptide, or an antibody to a particular splicing variant of the optineurin polypeptide); a ribozyme; a peptidomimetic; a small molecule or other agent that alters optineurin polypeptide activity and/or gene expression (e.g., which upregulate or downregulate expression of the optineurin gene); or another agent that alters (e.g., enhance or inhibit) optineurin gene expression or optineurin polypeptide activity, that alters posttranslational processing of the optineurin polypeptide, or that regulates transcription of optineurin splicing variants (e.g., agents that affect which splicing variants are expressed, or that affects the amount of each splicing variant that is expressed).

For example, an antibody to a mutant optineurin polypeptide can be used to inhibit an activity of the mutant protein. The term “antibody”, as used herein, refers to immunoglobulin molecules and immunologically active portions of immunoglobulin molecules, i.e., molecules that contain an antigen binding site that specifically binds an antigen. A molecule that specifically binds to a polypeptide is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. Either polyclonal or monoclonal antibodies can be used. The term “monoclonal antibody” or “monoclonal antibody composition”, as used herein, refers to a population of antibody molecules that contain only one species of an antigen binding site capable of immunoreacting with a particular epitope of a protein (e.g., the mutant optineurin). A monoclonal antibody composition thus typically displays a single binding affinity for a particular polypeptide with which it immunoreacts.

Polyclonal antibodies can be prepared as described above by immunizing a suitable subject with a desired immunogen, e.g., optineurin polypeptide or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds the protein of interest.

The many well known protocols used for fusing lymphocytes and immortalized cell lines can be applied for the purpose of generating a monoclonal antibody to an optineurin polypeptide (see, e.g., Current Protocols in Immunology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lemer (1981) Yale J. Biol. Med., 54:387-402. Moreover, the ordinarily skilled worker will appreciate that there are many variations of such methods that also would be useful.

Alternative to preparing monoclonal antibody-secreting hybridomas, a monoclonal antibody can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAPJ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J, 12:725-734.

Additionally, recombinant antibodies, such as chimeric and humanized monoclonal antibodies, comprising both human and non-human portions, which can be made using standard recombinant DNA techniques, can be used. Such chimeric and humanized monoclonal antibodies can be produced by recombinant DNA techniques known in the art.

Optineurin-Interacting Agents

In a fourth embodiment, the optineurin therapeutic agent can be a polypeptide which interacts with optineurin (“optineurin-interacting polypeptide”); a nucleic acid encoding such a polypeptide which interacts with optineurin; an agent which alters the expression or activity of optineurin-interacting polypeptide(s); and/or an agent which alters the interaction between optineurin and optineurin-interacting polypeptide(s). For example, as described below in detail and as shown in FIG. 2, optineurin may interact with proteins related to the FAS-ligand pathway and to the TNF-α pathway (e.g., Huntingtin, caspase 9), as well as to RAB-8, TFIIIA, and E3-14.7K. Optineurin additionally may interact with cytosolic phospholipase and cytochrome P450. Furthermore, optineurin appears to act through a feedback mechanism with TNF-α, and thereby play a neuroprotective role for optic neuropathies. Thus, alteration (increase or decrease) of the expression of any one of these polypeptides which interact with optineurin may alter the amount of activity of optineurin, and thereby can be used to enhance the neuroprotective role of optineurin. For example, in view of the proposed feedback mechanism of optineurin and TNF-α, and in view of the increase in TNF-α that is found in patients with glaucoma, an agent which controls TNF-α production or which decreases the amount of TNF-α may function in a similar manner to optineurin, that is, reducing TNF-α and thereby acting as a neuroprotectant against the effects of TNF-α. Thus, in a particular embodiment, the agent is an agent that alters expression of TNF-α Agents which alter the expression or activity of optineurin-interacting polypeptides can be, for example, one of the types of agents described herein (e.g., nucleic acids, polypeptides or proteins, antibodies, etc.).

More than one optineurin therapeutic agent can be used concurrently, if desired. The optineurin therapeutic agent(s) are administered in a therapeutically effective amount (i.e., an amount that is sufficient to treat the disease, such as by ameliorating symptoms associated with the disease, preventing or delaying the onset of the disease (e.g., particularly for an individual at increased risk for glaucoma), and/or also lessening the severity or frequency of symptoms of the disease). The amount which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The term, “treatment” as used herein, refers not only to ameliorating symptoms associated with the disease, but also preventing or delaying the onset of the disease, and also lessening the severity or frequency of symptoms of the disease. Thus, “treatment of glaucoma”, as used herein, refers not only to treatment after appearance of symptoms of glaucoma (therapeutic treatment), but also to prophylactic treatment (prior to appearance of symptoms). Treatment may be particularly beneficial for individuals in whom an increased risk of glaucoma has been identified, as it may delay onset of the disease, or prevent symptoms of the disease entirely. Thus, treatment can be used not only for individuals having glaucoma, but those at risk for developing glaucoma (e.g., those at increased risk for glaucoma, such as those having an alteration in the optineurin gene that is associated with increased risk of glaucoma).

In one embodiment of the invention, a nucleic acid is used in the treatment of glaucoma. The nucleic acid as described above can be used, either alone or in a pharmaceutical composition as described above. For example, the optineurin gene or a cDNA encoding the optineurin polypeptide, either by itself or included within a vector, can be introduced into cells (either in vitro or in vivo) such that the cells produce native optineurin polypeptide. In another example, a gene encoding an optineurin-interacting polypeptide or a cDNA encoding the optineurin-interacting polypeptide, either by itself or included within a vector, can be introduced into cells (either in vitro or in vivo) such that the cells produce native optineurin-interacting polypeptide. If necessary, cells that have been transformed with the gene or cDNA or a vector comprising the gene or cDNA can be introduced (or re-introduced) into an individual affected with the disease. Thus, cells which, in nature, lack native expression and activity of the polypeptide, or have mutant expression and activity, can be engineered to express the desired polypeptide (e.g., optineurin polypeptide, or, for example, an active fragment of the optineurin polypeptide). In a preferred embodiment, nucleic acid encoding the optineurin polypeptide, or an active fragment or derivative thereof, can be introduced into an expression vector, such as a viral vector, and the vector can be introduced into appropriate cells which lack native optineurin expression in an animal. For example, for the treatment of glaucoma, the vector comprising the nucleic acid can be introduced intraocularly. In such methods, a cell population can be engineered to inducibly or constitutively express active optineurin polypeptide. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid as described above, or a nucleic acid complementary to such a nucleic acid, can be used in “antisense” therapy, in which a nucleic acid (e.g., an oligonucleotide) which specifically hybridizes to the mRNA and/or genomic DNA of the optineurin gene (or to the mRNA and/or genomic DNA of a gene encoding an optineurin-interacting polypeptide) is administered or generated in situ. The antisense nucleic acid that specifically hybridizes to the mRNA and/or DNA can inhibit the expression of the optineurin polypeptide or of the optineurin-interacting polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA which is complementary to a portion of the mRNA and/or DNA which encodes optineurin polypeptide (or which encodes optineurin-interacting polypeptide). Alternatively, the antisense construct can be an oligonucleotide probe which is generated ex vivo and introduced into cells; it then inhibits expression by hybridizing with the mRNA and/or genomic DNA. In one embodiment, the oligonucleotide probes are modified oligonucleotides which are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al. ((1988) Cancer Res 48:2659-2668). With respect to antisense DNA, oligodeoxyribonucleotides derived from the translation initiation site, e.g. between the −10 and +10 regions of the optineurin gene sequence, are preferred.

To perform antisense therapy, oligonucleotides (mRNA, cDNA or DNA) are designed that are complementary to mRNA encoding optineurin (or encoding optineurin-interacting polypeptide). The antisense oligonucleotides bind to mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

Nucleic acid molecules to be used in triple helix formation for the inhibition of transcription are preferably single stranded and composed of deoxyribonucleotides. The base composition of these oligonucleotides should promote triple helix formation via Hogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex. The potential sequences that can be targeted for triple helix formation may be increased by creating a “switchback” nucleic acid molecule which is synthesized in an alternating 5′-3′,3′-5′ manner, such that they base pair with first one strand of a duplex and then the other, eliminating the necessity for a sizable stretch of either purines or pyrimidines to be present on one strand of a duplex.

In a preferred embodiment, oligonucleotides that are complementary to the 5′ end of the message, e.g., the 5′ untranslated sequence up to and including the AUG initiation codon, are used to inhibit translation. However, sequences complementary to the 3′ untranslated sequences of mRNAs have recently been shown to be effective at inhibiting translation of mRNAs as well (Wagner, R. (1994) Nature 372:333); therefore, oligonucleotides complementary to either the 5′ or 3′ untranslated, non-coding regions of the optineurin gene (or the gene encoding the optineurin-interacting polypeptide) can also be used in an antisense approach to inhibit translation of endogenous mRNA. Oligonucleotides complementary to the 5′ untranslated region of the mRNA can include the complement of the AUG start codon. Antisense oligonucleotides complementary to mRNA coding regions can also be used in accordance with the invention. While antisense nucleotides complementary to the can region sequence can be used, those complementary to the transcribed untranslated region can also be used. Whether designed to hybridize to the 5′, 3′ or coding region of optineurin mRNA, antisense nucleic acids are preferably at least six nucleotides in length, and are more preferably oligonucleotides ranging from 6 to about 50 nucleotides in length. In certain preferred embodiments, the oligonucleotide is at least 10 nucleotides, at least 18 nucleotides, at least 24 nucleotides, or at least 50 nucleotides.

If desired, in vitro studies can be performed to quantitate the ability of the antisense oligonucleotide to inhibit gene expression. These studies utilize controls that distinguish between antisense gene inhibition and nonspecific biological effects of oligonucleotides. These studies can compare levels of the target RNA or protein with that of an internal control RNA or protein. In a preferred embodiment, the control oligonucleotide is of approximately the same length as the test oligonucleotide and the nucleotide sequence of the oligonucleotide differs from the antisense sequence so much so as to prevent specific hybridization to the target sequence.

The oligonucleotides used in antisense therapy can be DNA, RNA, or chimeric mixtures or derivatives or modified versions thereof, preferably single-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al. (1989) Proc. Natl. Acad. Sci. USA 86:6553-6556; Lemaitre et al., (1987), Proc. Natl. Acad. Sci. USA 84:648-652; PCT International Publication No. WO88/09810) or the blood-brain barrier (see, e.g., PCT International Publication No. WO89/10134), or hybridization-triggered cleavage agents (see, e.g., Krol et al. (1988) BioTechniques 6:958-976) or intercalating agents. (See, e.g., Zon, (1988), Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide can comprise at least one (or more) modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. The antisense oligonucleotide can also comprise at least one modified sugar moiety selected from the group including but not limited to arabinose, 2-fluoroarabinose, xylulose, and hexose. In another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., (1987), Nucl. Acids Res. 15:6625-6641). The oligonucleotide is a 2′-O-methylribonucleotide (Inoue et al. (1987), Nucl. Acids Res. 15:6131-6148), or a chimeric RNA-DNA analogue (Inoue et al. (1987) FEBS Lett. 215:327-330).

Oligonucleotides can be synthesized by standard methods known in the art and described herein (e.g. by use of an automated DNA synthesizer (such as are commercially available from Biosearch, Applied Biosystems, etc.). As examples, phosphorothioate oligonucleotides can be synthesized by the method of Stein et al. ((1988) Nucl. Acids Res. 16:3209), methylphosphonate oligonucleotides can be prepared by use of controlled pore glass polymer supports (Sarin et al, (1988) Proc. Natl. Acad. Sci. USA. 85:7448-7451), etc.

The antisense molecules can be delivered to cells which express optineurin (or optineurin-interacting polypeptide) in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct can be utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct which can be introduced directly into the tissue site (e.g., the ocular tissue). Alternatively, viral vectors can be used which selectively infect the desired tissue, in which case administration may be accomplished by another route (e.g., systematically).

Ribozyme molecules designed to catalytically cleave optineurin mRNA transcripts can also be used to prevent translation of optineurin mRNA and expression of optineurin polypeptide, particularly, for example, to prevent translation of a mutant optineurin polypeptide. (See, e.g., PCT International Publication No. WO90/11364, and Sarver et al. (1990), Science 247:1222-1225). Alternatively, they can be designed to catalytically cleave mRNA transcripts of genes encoding optineurin-interacting polypeptides. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules includes one or more sequences complementary to the target gene mRNA, and must include the catalytic sequence responsible for mRNA cleavage. For this sequence, see U.S. Pat. No. 5,093,246. Specific ribozyme cleavage sites within any potential RNA target are initially identified by scanning the molecule of interest for ribozyme cleavage sites which include the following sequences, GUA, GUU and GUC. Once identified, short RNA sequences of between approximately 15 and 20 ribonucleotides corresponding to the region of the target gene containing the cleavage site may be evaluated for predicted structural features, such as secondary structure, that can render the oligonucleotide sequence unsuitable. The suitability of candidate sequences can also be evaluated by testing their accessibility to hybridization with complementary oligonucleotides, using ribonuclease protection assays. Ribozymes that cleave mRNA at site specific recognition sequences can be used to destroy optineurin mRNAs. In another embodiment, hammerhead ribozymes are used. Hammerhead ribozymes cleave mRNAs at locations dictated by flanking regions that form complementary base pairs with the target mRNA having the sequence of two bases: 5′-UG-3′. The construction and production of hammerhead ribozymes is described more fully in Haseloff and Gerlach, ((1988) Nature 334:585-591). Preferably the ribozyme is engineered so that the cleavage recognition site is located near the 5′ end of the optineurin mRNA, in order to increase efficiency and minimize the intracellular accumulation of non-functional mRNA transcripts.

The ribozymes used in the present invention can also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al (1984) Science 224:574-578; Zaug and Cech, (1986) Science 231:470-475; Zaug et al. (1986) Nature 324:429-433; PCT International Publication No. WO88/04300.; Been and Cech (1986) Cell 47:207-216). The Cech-type ribozymes have an eight base pair active site which hybridizes to a target RNA sequence, after which cleavage of the target RNA takes place. The invention further encompasses those Cech-type ribozymes which target eight base-pair active site sequences that are present in optineurin.

As in the antisense approach, the ribozymes can be composed of modified oligonucleotides (e.g. for improved stability, targeting, etc.) and can be delivered to cells which express optineurin in vivo (e.g., ocular cells). A preferred method of delivery involves using a DNA construct “encoding” the ribozyme under the control of a strong constitutive promoter, so that transfected cells will produce sufficient quantities of the ribozyme to destroy endogenous messages and inhibit translation. Because ribozymes unlike antisense molecules, are catalytic, a lower intracellular concentration is required for efficiency.

Endogenous optineurin gene expression, particularly mutant optineurin gene expression, can also be reduced by inactivating or “knocking out” the optineurin gene or its promoter, or the gene or promoter of an optineurin-interacting polypeptide, using targeted homologous recombination (e.g., see Smithies et al. (1985) Nature 317:230-234; Thomas & Capecchi (1987) Cell 51:503-512; Thompson et al. (1989) Cell 5:313-321). For example, a non-functional optineurin gene (or a completely unrelated DNA sequence) flanked by DNA homologous to the endogenous optineurin gene (either the coding regions or regulatory regions of the optineurin gene) can be used, with or without a selectable marker and/or a negative selectable marker, to transfect cells that express optineurin in vivo. Insertion of the DNA construct, via targeted homologous recombination, results in inactivation of the optineurin gene. Similar methods can be used for genes encoding optineurin-interacting polypeptides. The recombinant DNA constructs can be directly administered or targeted to the required site in vivo using appropriate vectors, as described above. Alternatively, expression of non-mutant optineurin or of optineurin-interacting polypeptides can be increased using a similar method: targeted homologous recombination can be used to insert a DNA construct comprising a non-mutant, functional gene in place of a mutant gene in the cell, as described above.

Alternatively, endogenous optineurin gene expression, or expression of a gene encoding an optineurin-interacting polypeptide, can be reduced by targeting deoxyribonucleotide sequences complementary to the regulatory region of the gene (i.e., the promoter and/or enhancers) to form triple helical structures that prevent transcription of the optineurin gene in target cells in the body. (See generally, Helene, C. (1991) Anticancer Drug Des., 6(6):569-84; Helene, C., et al. (1992) Ann, N.Y. Acad. Sci., 660:27-36; and Maher, L. J. (1992) Bioassays 14(12):807-15). Likewise, the antisense constructs, by antagonizing the normal biological activity of one of the optineurin polypeptides, can be used in the manipulation of tissue, e.g. tissue differentiation, both in vivo and for ex vivo tissue cultures. Furthermore, the anti-sense techniques (e.g. microinjection of antisense molecules, or transfection with plasmids whose transcripts are anti-sense with regard to a optineurin mRNA or gene sequence) can be used to investigate role of optineurin in developmental events, as well as the normal cellular function of optineurin in adult tissue. Such techniques can be utilized in cell culture, but can also be used in the creation of transgenic animals.

In yet another embodiment of the invention, polypeptides and/or agents that alter (e.g., enhance or inhibit) optineurin polypeptide activity, as described herein, can be used in the treatment or prevention of glaucoma. Polypeptides and/or agents that alter (e.g., enhance or inhibit) activity of optineurin-interacting polypeptides, as described herein, can also be used in the treatment or prevention of glaucoma. The polypeptides or agents can be delivered in a composition, as described above, or by themselves. They can be administered systemically, or can be targeted to a particular tissue (e.g., eye tissue). The proteins and/or agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-mutant optineurin polypeptide in conjunction with antisense therapy targeting mutant optineurin mRNA), can also be used.

Optineurin therapeutic agents can be administered to individuals to treat (prophylactically or therapeutically) glaucoma. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide, expression of a nucleic acid, or mutation content of the optineurin gene in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action”. Genetic conditions transmitted as single factors altering the way the body acts on drugs are referred to as “altered drug metabolism”. These pharmacogenetic conditions can occur either as rare defects or as polymorphisms.

Thus, the activity of an optineurin polypeptide, expression of an optineurin nucleic acid encoding the polypeptide, or mutation content of an optineurin gene in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as an optineurin therapeutic agent.

Compositions for Methods of Treatment

The methods of treatment described above utilize agents which can be incorporated into pharmaceutical compositions, if desired. For instance, a protein or protein, fragment, fusion protein or prodrug thereof, or a nucleotide or nucleic acid construct (vector) comprising a nucleic acid encoding optineurin, or an agent that alters optineurin activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. In a preferred embodiment, the composition is introduced intraocularly (e.g., eye drops). Other suitable methods of introduction can also include gene therapy (as described below), rechargeable or biodegradable devices, particle acceleration devises (“gene guns”) and slow release polymeric devices. The pharmaceutical compositions can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions that can be used in the methods of treatment. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Transgenic or Homologous Recombinant Animals

The invention also pertains to production of nonhuman transgenic animals. For example, in one embodiment, a host cell comprising a nucleic acid encoding optineurin (e.g., a fertilized oocyte or an embryonic stem cell into which a nucleic acid molecule encoding optineurin polypeptide) is used. Such host cells can be used to create nonhuman transgenic animals in which exogenous nucleotide sequences have been introduced into the genome or homologous recombinant animals in which endogenous nucleotide sequences have been altered. Alternatively, the invention also pertains to production of nonhuman animals in which the native optineurin has been altered.

Such animals are useful for studying the function and/or activity of the nucleotide sequence and polypeptide encoded by the sequence and for identifying and/or evaluating modulators of their activity, in order to investigate the processes of optineurin-associated glaucoma. As used herein, a “transgenic animal” is a nonhuman animal, preferably a mammal, more preferably a rodent such as a rat or mouse, or a primate, in which one or more of the cells of the animal includes a transgene. Other examples of transgenic animals include nonhuman primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a nonhuman animal, preferably a mammal, more preferably a mouse, in which an endogenous gene has been altered by homologous recombination between the endogenous gene and an exogenous DNA molecule (e.g., a mutated form of the endogenous gene) introduced into a cell of the animal, e.g. an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley (1991) Current Opinion in Bio/Technology, 2:823-829 and in PCT Publication Nos. WO 90/11354, WO 91/01140, WO 92/0968, and WO 93/04169. Clones of the nonhuman transgenic animals described herein can also be produced according to the methods described in Wilmut et al. (1997) Nature, 385:810-813 and PCT Publication Nos. WO 97/07668 and WO 97/07669.

The identification of optineurin gene's association with glaucoma, and its known interaction with a group of proteins, provides the first opportunity to examine biochemical pathways involved in the etiology of this group of eye disorders. Furthermore, identification of the gene as a significant contributing factor to the development of glaucoma allows screening for this disorder in high risk individuals, such as the elderly population, as well as prophylactic and therapeutic treatment of the disease.

The following Exemplification is offered for the purpose of illustrating the present invention and is not to be construed to limit the scope of this invention.

Exemplification: Identification of Optineurin and its Relationship to POAG

Family Materials

For mutation screening, a group of 54 adult-onset glaucoma families with a total of 147 living affected subjects was used, including a large family that was originally used to map the GLC1E locus to 10p14-p15 (Sarfarazi, M. et al., Am. J. Hum. Genet. 62:641 (1998)). The majority of these families presented only with LTG members (i.e., IOP≦22 mmHg), while others had mixed clinical manifestation of both LTG and moderately raised IOP (i.e., 23-26 mmHg) in different members. Additionally, 124 predominantly LTG and sporadic subjects were used for mutation screening of only one of the optineurin exons.

Identification of optineurin as the GLCIE-Causative Gene:

Direct sequencing of affected subjects was performed on an ABI-377 automated DNA Sequencer as described (see Stoilova, D. et al., J. Med. Genet. 35:989 (1998)). Sequencing was performed on samples from a published linked family (Sarfarazi, M. et al., Am. J. Hum. Genet. 62:641 (1998)). Initial screening of four candidate genes, IL2RA (interleukin 2 receptor alpha), IL15RA (interleukin 15 receptor alpha), GATA3 (GATA-binding protein 3) and NAPOR (neuroblastoma apoptosis-related RNA binding protein) did not identify any disease-causing mutations, although a number of silent (third base codon) changes, SNPs and insertion/deletion alterations were identified. A fifth gene was examined, and the sequencing identified a missense mutation (GAGB≧AAG; E50K) in exon 4 of optineurin (GenBank Accession #:AF420371 to AF420373; see also SEQ ID NO: 1, 3, 5). Sequencing of additional affected siblings and other more distantly affected relatives confirmed the presence of ESOK mutation in all of them (Table 1).

TABLE 1
Optineurin Significant Sequence Alterations

| | | | | | |
|---|---|---|---|---|---|
| | | | | # observed | |
| | | Predicted | # observed | mutations/ | |
| | | Protein | mutations/ | normal | |
| Exon | cDNA change | Change | families (%) | chromosomes | P values |

Disease-causing alterations

| | | | | | |
|---|---|---|---|---|---|
| 4 | GAG > AAG | E50K | 7/52 (13.5) | 0/540 | 2.74 × 10−8 |
| | (c458 G > A) | | | | |
| 6 | AG insertion | Premature | 1/46 (2.2) | 0/200 | 0.187 |
| | (c691-692 insAG) | stop | | | |
| 16 | CGG > CAG(c1944 | R545Q | 1/46 (2.2) | 0/100 | 0.315 |
| | G > A) | | | | |

| | | | |
|---|---|---|---|
| TOTALS | 9/54 (16.7) | 0.0 | 5.03 × 10^{-5*} |

Risk-associated alteration

| | | | | | |
|---|---|---|---|---|---|
| 5 | ATG > AAG(c603 | M98K | 23/169** | 9/422 (2.1) | 2.18 × 10−7 |
| | T > A) | | (13.6) | | |

*Only 100 shared chromosomes were used for calculation of this P value.
**Within the group of 169 subjects, M98K was observed in 8 of 45 (17.8%) familial and 15 of 124 (12.1%) sporadic individuals with glaucoma. Most of these individuals have normal IOP and were screened for sequence changes only in exon 5.72

Nucleotides are numbered as in GenBank accession number AF420371 (SEQ ID NO: 1). Normal chromosomes were from Caucasian individuals with a similar age group. P values are for Fisher's exact test. The 54 glaucoma families were screened by SSCP analysis for the entire optineurin gene.

Single strand conformational polymorphism (SSCP) assay of the E50K mutation perfectly segregated in this large family (49 members including 15 living affected). This mutation was absent in 540 normal control chromosomes. SSCP screening of 54 adult-onset glaucoma families identified the same ESOK mutation in another 6 pedigrees as well. The mutation segregated in 124 members, including 38 affected, 15 asymptomatic gene carriers, 50 unaffected, and 20 spouses. Therefore, it was concluded that E50K is a recurrent mutation. Of the 38 affected subjects with ESOK mutation, 7 (or 18.4%) had IOP measurements recorded between 23-26 mmHg while the remaining individuals had IOP values ranging between 11-21 mmHg.

Two additional mutations (2-base pair AG insertion, and R545Q) were identified in two other families with normal IOP (Table 1).

The second mutation in exon 6 (2-bp “AG” insertion after ASP127) was observed in a LTG subject. This mutation shifts the open reading frame after the point of insertion and translates into 22 new amino acids before finally terminating with a new premature stop codon. This truncates the protein at 76% of the normal protein.

The third mutation in exon 16 (CGG>CAG; R545Q) was identified in another unrelated LTG subject. This mutation was not present in over 100 normal chromosomes.

A fourth sequence change in exon 5 (ATG>AAG; M98K) was documented in 23 (or 13.6%) out of a total of 169 index cases (i.e., 45 families and 124 other sporadic and predominantly LTG subjects screened only for this exon). Only three of these 23 subjects had IOP values recorded above normal average (i.e., 23, 26 and 40 mmHg) while the remaining 20 subjects were previously diagnosed as LTG. The M98K change was also present in 9 out of 422 (or 2.1%) normal control chromosomes; however, these nine subjects did not receive a comprehensive glaucoma examination and, therefore, it is likely that one or more of them will eventually develop glaucoma. Nevertheless, since the observed difference between affected (13.6%) and normal control (2.1%) frequencies is highly significant (X2=30.99; df=1; P=2.18×10⁻⁷), and as the altered amino acid is also conserved in macaque (see below), the M98K indeed represents a risk-associated factor for glaucoma.

Taken together, sequence alterations in the optineurin gene may be responsible for 16.67% to 17.98% (32 out of 178) of adult-onset glaucoma (see Table 1, above). Additional mutations in the families and/or sporadic cases may also be present. Genotyping and inspection of a number of flanking DNA and optineurin intragenic markers did not identify a common haplotype in these 7 families.

Additional Sequence Alterations in Optineurin:

Eight additional sequence alterations were identified (see Table 2). These changes were verified by sequencing genomic DNA, BAC clones and cDNAs prepared from human trabecular meshwork (HTM) and lymphocytes. The observed changes were found to be consistent in all of our samples but different from those for FIP-2 (Accession # AF061034).

TABLE 2
Optineurin Polymorphisms and Sequence Alterations

| | | Amino Acid |
|---|---|---|
| Exon | cDNA Change | Change |
| 4 | ACG > ACA | T34T |
| | (c412 G > A) | |
| 4 | CTG > CTA | L41L |
| | (c433 G > A) | |
| 6 | GAA > GAG | E163E |
| | (c799A > G) | |
| 7 | TCC > CCC | S201P |
| | (c911 T > C) | |
| 8 | c947 C > A | H213K |
| 8 | c949 C > A | H213K |
| 8 | AGG > AGC | R216S |
| | (c958 G > C) | |
| 11 | CCT > ACT | P357T |
| | (c1379 C > A) | |

Nucleotides are numbered as in GenBank accession number AF420371 (SEQ ID NO: 1).

Several other alterations in the optineurin gene have been identified by similar methods as the mutations described in Tables 1 and 2. These alterations, shown in Table 3 below, may be either DNA polymorphisms, or may be associated with the presence of glaucoma.

TABLE 3
Other Optineurin Sequence Alterations

| | | Amino Acid |
|---|---|---|
| Exon | cDNA Change | Change |
| 4 | CCC > GCC | P16A |
| | c356C > G | |
| 4 | CAG > CAC | Q42H |
| | c432G > C | |
| 5 | GAA > GTA | E92V |
| | c585A > T | |
| 8 | CAG > CAA | Q245Q |
| | c1045G > A | |
| 10 | AGC > AGT | S297S |
| | C1201C > T | |
| 10 | GAA > AAA | E322K |
| | C1274G > A | |

Nucleotides are numbered as in GenBank accession number AF420371 (SEQ ID NO: 1).

Optineurin Genomic and Protein Structure:

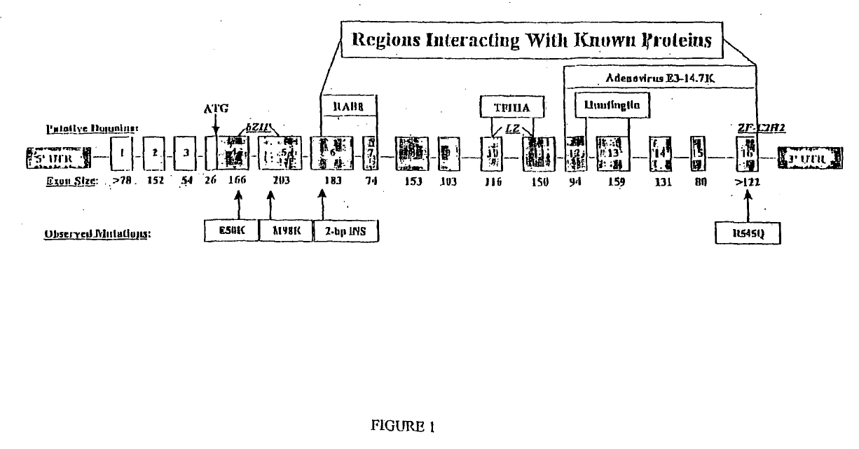

Optineurin maps to the GLC1E locus and narrows down its physical location to 10p14. As shown in FIG. 1, this gene contains 3 non-coding exons at its 5′-untranslated region (UTR) and another 13 exons that encode for a total of 577 amino acids (aa). In FIG. 1, approximate regions interacting with other known proteins are indicated, as are putative functional domains, sizes of each exon, and position and type of mutations observed. Splicing was identified at the 5′-UTR that generated at least 3 different isoforms (Accession # AF420371-3) but none has altered the coding exons.

Optineurin is a cellular protein that contains two putative bZIP transcription factor basic motifs, several leucine-zipper domains and a C2H2 type zinc finger (FIG. 1). This acidic protein (pI=5.15) is rich in both glutamate (15.8%) and leucine (11.8%).

Optineurin in Other Species:

During this study, the mouse and optineurin gene was also cloned (SEQ ID NOs:9, 10). The mouse gene encodes for 584 aa (67 kDa) and shows 78% identity to human optineurin. The mouse gene also divides into 13 coding exons and its boundaries are fully conserved with human. Also cloned was a complete cDNA sequence for optineurin in crab-eating macaque (571 aa; 65 kDa) (SEQ ID NOs: 11-14). Inspection of public databases also identified and other partial sequences for rat (Moreland, R. J. et al., Nucleic Acids Res. 28:1986 (2000)) (SEQ ID NO: 15,16), pig (SEQ ID No: 17,18) and bovine. Overall, human optineurin has 78%-85% identity with its homologs in mouse, rat, pig and bovine, and 96% identity with macaque. Interestingly, both E50K and M98K mutations observed respectively in 7 and 23 index cases of this study are conserved between human and macaque. The M98K evolutionary conservation further corroborates that this mutation is a risk factor for glaucoma. The E50K mutation is further conserved in mouse and bovine.

Ocular and Non-Ocular Expression of Human Optineurin:

By PCR amplification, expression of optineurin was shown in samples prepared from human trabecular network HTM, non-pigmented ciliary epithelium (NPCE), retina, brain, adrenal cortex, liver, fetus, lymphocyte and both normal (NHDF) and mutant (E50K-DF) dermal fibroblasts. Northern analysis was performed with an optineurin-specific cDNA probe (approximately 2.0 kb) that was radioactively labeled and hybridized with 5 micrograms of polyA+ RNA from two human cell lines, established from (1) trabecular meshwork and (2) non-pigmented ciliary epithelium. By the Northern blotting, a major band of approximately 2.0 kb message was documented in both HTM and NPCE cell lines; it was 3-4 times more abundant than a 3.6 kb message. These transcripts are in general agreement with previously reported message in heart, brain, placenta, liver, skeletal muscle, kidney and pancreas (Li, Y. et al., Mol. Cell. Biol. 18:1601 (1998)).

Western Analysis

cDNA sequence alignments of human, macaque, mouse, rat, pig and bovine showed a significant degree of protein conservation across these species. In addition, selected anti-peptide antibodies were prepared. Two different 18-amino acid peptides from the N-terminus (MSHQPLSCLTEKEDSPSE, SEQ ID NO:7) and C-terminus (EVLPDIDTLQIHVMDCII, SEQ ID NO: 8) of optineurin were used to immunize chicken and to obtain anti-optineurin antibodies.

Standard ELISA, immunoblotting and immunocytochemistry assays were developed using the anti-optineurin antibodies. Specificity of the antibodies in these assays was documented by two separate methods: first, non-immune immunoglobulin was used as a specificity control, which did not react in any experiments conducted during the investigation; second, the anti-human optineurin antibody was preadsorbed with optineurin-specific peptide antigen. The preadsorption abolished all immunoreactivity in the cells tested.

These selected anti-peptide antibodies were 100% conserved in the known sequences of both mouse and macaque optineurin. Western analysis was performed using a variety of cell types. Cells were washed with ice-cold protease inhibitor buffer (one tablet of Roche protease inhibitor cocktail in 10 ml of Phosphate Buffered Saline (PBS)). Cells were lysed by adding protease inhibitor buffer and supplemented with 1% CHAPS. Cell lysates (approximately 40 μg of protein per lane) were subjected to 4-15% Tris-HCl gradient gels and blotted onto PVDF membranes. Nonspecific hybridization was blocked with 5% skim dry milk in PBST (PBS with 0.5% Tween 20). Membrane was probed with primary antibody (human optineurin anti-peptide antibody, raised in chicken as described above) at 1/100. After washing, membranes were incubated with secondary antibody (rabbit anti-chicken conjugated with HRP) at 1:10,000 (Sigma). Colorimetric detection was carried out using Opti-4CN Kit (Bio-Rad). One of these antibodies cross-reacted with an approximately 66-kDa protein in whole cell extracts from different lines including HTM, NPCE, ESOK-EF, NHDF and HeLa.

Because optineurin was detected in both HTM and NPCE, and, as the latter is a component of transport and secretory epithelium, we decided to determine optineurin expression in aqueous humor. For this purpose, a Zoo Western blot was prepared from aqueous humors of human and 7 other species. Since cloning of the mouse gene predicted a protein size of 67 kDa, NIH3T3 cell line was used as another control. All samples showed presence of similar sized proteins, including those based on the known sequences of human (66 kDa), macaque (65 kDa) and mouse optineurins (67 kDa). The presence of this protein was further confirmed in eye tissue homogenates prepared from a selected group of these animals. These data indicate that optineurin is a secretory protein that is highly conserved during evolution.

Immunocytochemical Analysis of Optineurin:

Both primary (NHDF and E50K-DF) and transformed (HTM and NPCE) cell lines were used to study cellular localization of the protein. An immunocytochemistry study demonstrated granular staining for optineurin endogenous protein that is associated with vesicular structures near the nucleus. Cells were seeded in 6-well plates and grown on glass coverslips for 48 hours and the medium was changed once after 24 hours. Cells were then washed twice in PBS, fixed in 4% Paraformaldehyde for 20 minutes on ice, rinsed twice in PBS and permeablized in 0.1% Triton X-100 for 10 minutes. After two washes in PBS, nonspecific hybridization was blocked with 4% bovine serum albumin in PBS for 30 minutes. Cells were incubated with primary antibody at 1/200 for one hour. After washing, cells were incubated with secondary antibody (goat anti-chicken, Molecular Probes, Inc.) and labeled with Alexa Fluor 488 (Green) or Alexa Fluor 594 (Red) at 1/500 for 45 minutes. For nucleic acid staining, after washing, cells were incubated with TO-PRO-3 iodide at 1/200 for 30 minutes. For Golgi Apparatus staining, cells were incubated with BODIPY FL C5-ceramide at 1/3000 for 30 minutes. After washing in PBS, coverslips were mounted on slides with antifade reagent, and examined using Zeiss 410 Laser Scanning Confocal Microscope.

There was a consistent perinuclear localization pattern for the endogenous protein in both virally transformed and in non-transformed, normal cell lines. Specific staining for Golgi indicated a perinuclear localization of this protein that extends to structures on the Golgi complex and on vesicles. As a control, use of both non-immune immunoglobulin and optineurin-specific peptide antigen was used. They did not react with any of the cell types.

During the study it was noted that although both normal and E50K mutant fibroblast cultures grew naturally and equally, the amount of endogenous protein product in the E50K mutant cells was substantially lower than in the normal cells—it was either completely negative or very weakly positive for optineurin. Furthermore, only 10-20% of the E50K cells were positive for the optineurin polypeptide, as compared to 70-80% of the normal cells. Additionally, within the very limited E50K positive cells, optineurin polypeptide appeared to be less perinuclear and more disorganized. Therefore, the effect of E50K optineurin mutation appears to both lower synthesis and redistribute protein products in the affected cells. In certain other cells examined, optineurin was poorly detectable in the cytoplasm. The predicted instability of this protein, together with its heterogeneous intracellular distribution, suggests that optineurin is expressed transiently as it either is rapidly secreted out of the mature cells, or is removed from the mature cells, probably through degradation signals in its 3′ UTR. This prediction is supported by the fact that with time, concentration of this protein accumulated in the cell culture medium at much higher levels than observed intracellularly.

Discussion

Three disease-causing alterations in the optineurin gene have been identified in nine adult-onset, low tension glaucoma (LTG)/POAG families, and a risk-associated alteration in 23 (primarily) LTG index cases (see Table 1, above). A conservative estimate indicates that mutations in this gene are responsible for 16.67% to 17.98% of all glaucoma patients studied (see Table 1). Since there are up to 1.2 million LTG and up to 2.47 million POAG subjects in the United States alone, screening for optineurin mutations could detect over 200,000 cases of LTG and up to 440,000 cases of POAG. Perhaps as many as twice this number of individuals are already affected with this condition without any identifiable clinical signs or symptoms. A recurrent mutation (E50K) that is also conserved in mouse, bovine and macaque was identified in the basic region of the first putative bZIP transcription factor domain. Since bZrP domains have a basic region for sequence-specific DNA binding, it appears that E40K is abrogating this potential DNA-binding capability of optineurin. A second mutation, an “AG” insertion in exon 6 (after Aspl27), was found that shifted the open reading frame and truncated the normal protein by 75%. This truncated protein is expected to forfeit the normal interaction of optineurin with RAB8, TFIIIA, Huntingtin and E3-14.7K proteins (see FIG. 1). A third mutation (R545Q) was identified in exon 16. Although this mutation is not part of a known protein domain, it is close to the only C₂H₂ zinc finger motif in the optineurin molecule. Since such a domain is usually found in transcription factors, it is likely that the observed mutation interferes with this potential function of optineurin. Another alteration (M98K) was observed in exon 5 of optineurin; this alteration is present in 13.61% of mainly LTG index cases, and 2.13% of normal controls (p<0.00001). As this sequence change was located within the second putative bZIP transcription factor basic domain (see FIG. 1), and is also conserved in macaque, it appears to be another risk factor for this condition.

Optineurin is not known to have any significant homology to any known protein, to date; however, its interaction with a number of other proteins has been hypothesized. FIG. 2 provides a pictorial illustration of possible optineurin interactions with other proteins and its potential involvement in alternative pathways. Potential involvement of optineurin in two alternative pathways of FAS-Ligand (left) and TNF-α (right) are shown; interactions are depicted with solid arrows and downstream effects with open arrows. Arrows ending with a circle depict the blocking effect of one protein on another. It has previously been reported that adenovirus E3-14.7K interacts with the last 172 amino acids of optineurin (Li, Y. et al., Mol. Cell. Biol. 18:1601 (1998)). This specific interaction can block the protective effect of E3-14.7K on TNF-α cell killings induced by its receptors (i.e., TNFR1 and RIP). TNF-α can also directly induce optineurin expression in a time-dependent manner (id.). This suggests that optineurin is a component of the TNF-α signaling pathway that can shift the equilibrium toward the induction of apoptosis. Furthermore, it has been documented that TNF-α markedly increases the severity of damage in optic nerve heads of POAG/LTS subjects (Yuan, L. and Neufeld, A. H., Glia 32:42 (2000); Tezel, G. and Wax, M. B., J. Neurosci. 20:8694 (2000)). Generation of this cytokine by reactive optic nerve head astrocytes and glial cells can induce excessive nitric oxide and drive them to be neurotoxic to the axons of the retinal ganglion cells (id.). Therefore, it appears that normal endogenous optineurin, either directly or through its interaction with other proteins, can restrain TNF-α production, possibly through a feedback mechanism, and thereby play a neuroprotective role for this group of optic neuropathies. Consequently, the mutant forms of optineurin in glaucoma patients may provide an inadequate neuroprotection over decades of normal life, thus leading to the late-onset presentation of this optic neuropathy.

As shown in FIG. 2, the TNF-α pathway includes activation of cytosolic phospholipase A2 (cPLA2) to release arachidonic acid (AA) and its potent products, the mediators of inflammation (Wold, W. S., J. Cell Biochem. 53:329 (1993)). As E3-14.7K can block this inflammatory response (id.), it appears that optineurin interaction with this protein may also reverse its blocking ability. Therefore, optineurin involvement in TNF-α pathway could potentially lead to either apoptosis or inflammation. In a third alternative pathway, Cytochrome P450 can metabolize AA into biologically active molecules that may be directly relevant to the glaucoma phenotype. In support of this, it has previously been shown that mutations in Cytochrome P4501B1 are responsible for primary congenital glaucoma (Stoilov, I. et al., Hum. Mol. Genet. 6:641 (1997); Stoilov, I. et al., Am. J. Hum. Genet. 62:573 (1998)). One AA metabolite that is directly implicated in blood vessel constriction and ion transport (McGiff, J. C. et al., Curr. Opin. Nephrol. Hypertens. 10:231 (2001)) is 20-hydroxyeicosatetraenoic acid (20-HETE). Since recurrent vasospasm is frequently reported in LTG patients (Gasser, P. et al., Angiology 41:214 (1990); Rankin, S. J., Surv. Ophthalmol. 43 Suppl 1:S176 (1999)), and since vasoconstriction leads to reduced aqueous humor production (Van Buskirk, E. M. et al., Am. J. Ophthalmol. 109:511 (1990)), it appears that optineurin mutations through AA-P450 pathway plays a role in the structural damage reported in LTG patients (Caprioli, J. and Spaeth, G. L., Am. J. Ophthalmol. 97:730 (1984)). The effectiveness of calcium-channel-blockers in the treatment of LTG (Netland, P. A. et al., Am. J. Ophthalmol. 115:608 (1993)) and an observed optineurin polypeptide expression in human coronary arterial cell cultures further support this theory. Vasospasm is not only present in LTG, but also in Raynaud's disease and migraine (Gasser, P. et al., Angiology 41:214 (1990)), two conditions frequently reported with high pressure POAG.

Since E3-14.7K interaction with Caspase-8 (CASP8) can efficiently block Fas Ligand-induced apoptosis (Chen, P. et al., J. Biol. Chem. 273:5815 (1998)), it appears that optineurin either forms a protein complex with E3-14.7K and CASP8 to inhibit apoptosis, or alternatively as previously reported for TNF-α (Li, Y. et al., Mol. Cell. Biol. 18; 1601 (1998)), this interaction may reverse the protective effect of E3-14.7K and thereby induce apoptosis. Therefore, interaction of optineurin with E3-14.7K may regulate signaling pathways downstream of both TNF receptors and Fas.

In addition to E3-14.7K, the C-terminal part of optineurin also interacts with Huntingtin (Faber, P. W. et al., Hum. Mol. Genet. 7:1463 (1998)), the defective protein in Huntington Disease (HD). Huntingtin is reported to have an anti-apoptotic effect (Wellington, C. L. et al., J Neural. Transm. Suppl. 1(2000)). Since Huntingtin and E3-14.7K both bind to the C-terminal of optineurin, it is possible that binding of Huntingtin to optineurin would neutralize apoptotic signals normally mediated through optineurin (Li, Y. et al., Mol. Cell. Biol. 18:1601 (1998)). Likewise, E3-14.7K interacts with CASP8 to inhibit FAS Ligand-induced apoptosis (Chen, P. et al., J. Biol. Chem. 273:5815 (1998)). Since CASP8 is required for cell death induced by expanded polyglutamine repeats in HD (Sanches, I. et al., Neuron 22:623 (1999)), a potential multidimensional protein complex formation between optineurin-huntingtin-CASP8-E3-14.7K may play a common role in neurodegeneration of both HD and POAG. Furthermore, optineurin also indirectly links Huntingtin to RAB8 (Hattula, K. and Peranen, J., Curr. Biol. 10:1603 (2000)), a small GTPase protein that binds to the N-terminal region of optineurin. Since reorganization of actin and microtubules by RAB8 dictate drastic changes in the cell shape, it is likely that a complex molecule formed by interaction of RAB8-optineurin-Huntingtin plays a central role in controlling cellular morphogenesis, membrane (through RAB8) or vesicle (through Huntingtin) trafficking. The immunocytochemistry localization of optineurin to the Golgi apparatus suggests that protein trafficking is a function for this molecule. Recently, in a new Xenopus transgenic model it was shown that mutant forms of RAB8 protein cause retinal degeneration (Moritz, O. et al., Mol. Biol. Cell 12:2341 (2001)), and that this protein is involved in docking of post-Golgi membranes in rods (id.).

The central leucine-rich domain of optineurin (FIG. 1) interacts with the N-terminal portion of TFIIIA (Moreland, R. J. et al., Nucleic Acids Res. 28:1986 (2000)). The latter binds to the internal control region of 5S ribosomal DNA and then, in association with TFIIIB and TFIIIC, forms a stable preinitiation complex for gene transcription by RNA polymerase III. It is likely that optineurin interaction with TFIIIA transforms this molecule from an inactive to an inactive state and thereby activates its transcription.

Optineurin has also been cloned as a NEMO (NF-κB Essential Modulator or FIP3)-related protein (NRP) but shown to have no effect on NF-κB signaling (Schwambom, K. et al., J. Biol. Chem. 275:22780 (2000)). Phorbol esters induced optineurin phosphorylation but at the same time reduced its half-life (id.). This phosphorylation was reported not to affect the subcellular localization of endogenous optineurin (id.). Although no specific kinase activity responsible for this phosphorylation has been identified, the authors showed that optineurin could function in the assembly and activity of two unknown kinases with molecular weight of 85- and 180-kDA.

The teachings of the references cited herein are incorporated by reference in their entirety.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. An isolated nucleic acid molecule comprising SEQ ID NO: 1, an oligonucleotide of about 10 to about 50 nucleotides of SEQ ID NO: 1, SEQ ID NO: 3, an oligonucleotide of about 10 to about 50 nucleotides of SEQ ID NO: 3, SEQ ID NO: 5, an oligonucleotide of about 10 to about 50 nucleotides of SEQ ID NO: 5, or a complement of one of the foregoing nucleic acid molecules, wherein the nucleic acid molecule has an alteration in at least one nucleotide, and wherein the alteration is indicative of the presence of an optineurin-associated glaucoma or of an optineurin-associated risk of glaucoma.

2. The isolated nucleic acid molecule of claim 1, wherein the alteration produces a sequence change in a polypeptide encoded by the nucleic acid molecule.

3. The isolated nucleic acid molecule of claim 2, wherein the alteration is a change from GAG to AAG at codon 50, an insertion of AG after codon 127, a change from CGG to CAG at codon 545, a change from ATG to AAG at codon 98, a change from CCC to GCC at codon 16, a change from CAG to CAC at codon 42, a change from GAA to GTA at codon 92, a change from GAA to AAA at codon 322, a complement of one of the foregoing alterations, or a combination comprising one or more of the foregoing alterations.

4. The isolated nucleic acid of claim 1, wherein the glaucoma is primary open angle glaucoma.

5. A purified polypeptide comprising SEQ ID NO: 2, a peptide of about 10 to about 50 contiguous amino acids of SEQ ID NO: 2, SEQ ID NO: 4, a peptide of about 10 to about 50 contiguous amino acids of SEQ ID NO: 4, SEQ ID NO: 6, or a peptide of about 10 to about 50 contiguous amino acids of SEQ ID NO: 6, wherein the polypeptide has an alteration in at least one amino acid, and wherein the alteration is indicative of the presence of an optineurin-associated glaucoma or of an optineurin-associated risk of glaucoma.

6. The purified polypeptide of claim 5, wherein the alteration is a change from glutamic acid to lysine at codon 50, a premature stop after codon 127, a change from arginine to glutamine at codon 545, a change from methionine to lysine at codon 98, a change from proline to alanine at codon 16, a change from glutamine to histidine at codon 42, a change from glutamic acid to valine at codon 92, a change from glutamic acid to lysine at codon 322 or a combination comprising one or more of the foregoing alterations.

7. The purified polypeptide of claim 5, wherein the glaucoma is primary open angle glaucoma.

8. An array of nucleic acid molecules attached to a solid support, the array comprising an oligonucleotide comprising about 10 to about 50 nucleotides of SEQ ID NO: 1, about 10 to about 50 nucleotides of SEQ ID NO: 3, about 10 to about 50 nucleotides of SEQ ID NO: 5, or a complement of one of the foregoing oligonucleotides, wherein the oligonucleotide has an alteration in at least one nucleotide, and wherein the alteration is indicative of the presence of an optineurin-associated glaucoma or of an optineurin-associated risk of glaucoma.

9. The array of claim 8, wherein the alteration produces a sequence change in a polypeptide encoded by an optineurin nucleic acid molecule comprising the oligonucleotide.

10. The array of claim 9, wherein the alteration is a change from GAG to AAG at codon 50, an insertion of AG after codon 127, a change from CGG to CAG at codon 545, a change from ATG to AAG at codon 98, a change from CCC to GCC at codon 16, a change from CAG to CAC at codon 42, a change from GAA to GTA at codon 92, a change from GAA to AAA at codon 322, a complement of one of the foregoing alterations, or a combination comprising one or more of the foregoing alterations.

11. A method of detecting the presence or absence of an optineurin-associated glaucoma or an optineurin-associated risk of glaucoma in a sample from an individual, comprising assessing the sample for:
an alteration in an optineurin nucleic acid; or
an alteration in an optineurin polypeptide;
wherein the alteration in the optineurin nucleic acid or the alteration in the optineurin polypeptide is indicative of the presence or the absence of an optineurin-associated glaucoma or an optineurin-associated risk of glaucoma.

12. The method of claim 11, wherein the alteration is in an optineurin nucleic acid.

13. The method of claim 12, wherein the optineurin nucleic acid comprises at least a fragment of about 10 to about 50 nucleotides of SEQ ID NO: 1, SEQ ID NO: 3, or SEQ ID NO: 5.

14. The method of claim 13, wherein the alteration is a change from GAG to AAG at codon 50, an insertion of AG after codon 127, a change from CGG to CAG at codon 545, a change from ATG to AAG at codon 98, a change from CCC to GCC at codon 16, a change from CAG to CAC at codon 42, a change from GAA to GTA at codon 92, a change from GAA to AAA at codon 322, or a combination comprising one or more of the foregoing alterations.

15. The method of claim 12, wherein assessing comprises sequencing at least a portion of a optineurin nucleic acid, or hybridizing a nucleic acid probe to a nucleic acid.

16. The method of claim 12, wherein assessing comprises utilizing an array of nucleic acid molecules attached to a solid support.

17. The method of claim 16, wherein at least one of the nucleic acid molecules is an oligonucleotide having an alteration in at least one nucleotide, and wherein the alteration is indicative of the presence of optineurin-associated glaucoma or of an optineurin-associated risk of glaucoma.

18. The method of claim 11, wherein the alteration is in an optineurin polypeptide.

19. The method of claim 18, wherein the alteration is a change from glutamic acid to lysine at codon 50, a premature stop after codon 127, a change from arginine to glutamine at codon 545, a change from methionine to lysine at codon 98, a change from proline to alanine at codon 16, a change from glutamine to histidine at codon 42, a change from glutamic acid to valine at codon 92, a change from glutamic acid to lysine at codon 322, or a combination comprising one or more of the foregoing alterations.

20. The method of claim 11, comprising detecting an alteration in an optineurin nucleic acid or an optineurin polypeptide associated with the presence of optineurin-associated glaucoma.

21. The method of claim 20, further comprising diagnosing optineurin-associated glaucoma in an individual.

22. The method of claim 20, wherein the alteration is a change from GAG to AAG at codon 50, an insertion of AG after codon 127, a change from CGG to CAG at codon 545, or a combination comprising one or more of the foregoing alterations.

23. The method of claim 11, comprising detecting an alteration in an optineurin nucleic acid or an optineurin polypeptide associated with the presence of an optineurin-associated risk of glaucoma.

24. The method of claim 23, further comprising diagnosing an optineurin-associated risk of glaucoma in an individual.

25. The method of claim 23, further comprising prognosing an optineurin-associated risk of glaucoma in an individual.

26. The method of claim 23, wherein the alteration is a change from ATG to AAG at codon 98.

27. The method of claim 11, comprising detecting the absence of an alteration in an optineurin nucleic acid and diagnosing the absence of an optineurin-associated glaucoma, an optineurin-associated risk of glaucoma, or both.

28. The method of claim 11, wherein the glaucoma is a primary open angle glaucoma.

29. A method of diagnosing glaucoma or the risk of glaucoma in an individual comprising assessing a sample from the individual for an alteration in an optineurin nucleic acid, an alteration in an optineurin polypeptide, or both.

30. The method of claim 29, wherein the optineurin nucleic acid comprises at least a fragment of about 10 to about 50 nucleotides of SEQ ID NO: 1, SEQ ID NO: 3, or SEQ ID NO: 5.

31. The method of claim 30, wherein the alteration is a change from GAG to AAG at codon 50, an insertion of AG after codon 127, a change from CGG to CAG at codon 545, a change from ATG to AAG at codon 98, a change from CCC to GCC at codon 16, a change from CAG to CAC at codon 42, a change from GAA to GTA at codon 92, a change from GAA to AAA at codon 322, or a combination comprising one or more of the foregoing alterations.

32. The method of claim 29, wherein the optineurin polypeptide comprises at least an active fragment of SEQ ID NO: 2, SEQ ID NO: 4, or SEQ ID NO: 6.

33. The method of claim 32, wherein the alteration is a change from glutamic acid to lysine at codon 50, a premature stop after codon 127, a change from arginine to glutamine at codon 545, a change from methionine to lysine at codon 98, a change from proline to alanine at codon 16, a change from glutamine to histidine at codon 42, a change from glutamic acid to valine at codon 92, a change from glutamic acid to lysine at codon 322, or a combination comprising one or more of the foregoing alterations.

34. A method of screening for glaucoma or the risk of glaucoma in an individual comprising assessing a sample from the individual for an alteration in an optineurin nucleic acid, an alteration in an optineurin polypeptide, or both.

35. The method of claim 34, wherein the optineurin nucleic acid comprises at least a fragment of about 10 to about 50 nucleotides of SEQ ID NO: 1, SEQ ID NO: 3, or SEQ ID NO: 5.

36. The method of claim 35, wherein the alteration is a change from GAG to AAG at codon 50, an insertion of AG after codon 127, a change from CGG to CAG at codon 545, a change from ATG to AAG at codon 98, a change from CCC to GCC at codon 16, a change from CAG to CAC at codon 42, a change from GAA to GTA at codon 92, a change from GAA to AAA at codon 322, or a combination comprising one or more of the foregoing alterations.

37. The method of claim 34, wherein the optineurin polypeptide comprises at least an active fragment of SEQ ID NO: 2, SEQ ID NO: 4, or SEQ ID NO: 6.

38. The method of claim 37, wherein the alteration is a change from glutamic acid to lysine at codon 50, a premature stop after codon 127, a change from arginine to glutamine at codon 545, a change from methionine to lysine at codon 98, a change from proline to alanine at codon 16, a change from glutamine to histidine at codon 42, a change from glutamic acid to valine at codon 92, a change from glutamic acid to lysine at codon 322, or a combination comprising one or more of the foregoing alterations.

39. A method of treating glaucoma in an individual, comprising administering to the individual an optineurin therapeutic agent in a therapeutically effective amount.

40. A method of treating an individual having an increased risk for glaucoma, comprising administering to the individual an optineurin therapeutic agent in a therapeutically or prophylacticly effective amount.

41. A computer readable medium comprising an optineurin nucleic acid sequence, an optineurin polypeptide sequence, or a combination thereof,
wherein the optineurin nucleic acid sequence comprises SEQ ID NO: 1, an oligonucleotide of about 10 to about 50 nucleotides of SEQ ID NO: 1, SEQ ID NO. 3, an oligonucleotide of about 10 to about 50 nucleotides of SEQ ID NO: 3, SEQ ID NO: 5, an oligonucleotide of about 10 to about 50 nucleotides of SEQ ID NO: 5, or the complement of one of the foregoing nucleic acid sequences, wherein the nucleic acid sequence has an alteration in at least one nucleotide, and wherein the alteration is indicative of the presence or absence of optineurin-associated glaucoma or of an optineurin-associated risk of glaucoma; and
wherein the optineurin polypeptide sequence comprises SEQ ID NO: 2, a peptide of about 10 to about 50 contiguous amino acids of SEQ ID NO: 2, SEQ ID NO. 4, a peptide of about 10 to about 50 contiguous amino acids of SEQ ID NO: 4, SEQ ID NO: 6, or a peptide of about 10 to about 50 contiguous amino acids of SEQ ID NO: 6, wherein the polypeptide sequence has an alteration in at least one amino acid, and wherein the alteration is indicative of the presence or absence of optineurin-associated glaucoma or of an optineurin-associated risk of glaucoma.

42. The computer readable medium of claim 41, wherein the optineurin nucleic acid sequence comprises a change from GAG to AAG at codon 50, an insertion of AG after codon 127, a change from CGG to CAG at codon 545, a change from ATG to AAG at codon 98, a change from CCC to GCC at codon 16, a change from CAG to CAC at codon 42, a change from GAA to GTA at codon 92, a change from GAA to AAA at codon 322, a complement of one of the foregoing alterations, or a combination comprising one or more of the foregoing alterations.

43. The computer readable medium of claim 41, wherein the optineurin polypeptide sequence comprises a glutamic acid to lysine at codon 50, a premature stop after codon 127, an arginine to glutamine at codon 545, a methionine to lysine at codon 98, a proline to alanine at codon 16, a glutamine to histidine at codon 42, a glutamic acid to valine at codon 92, a glutamic acid to lysine at codon 322. or a combination comprising one or more of the foregoing alterations.
